(19) United States
(12) Patent Application Publication
(10) Pub. No.: US 2022/0395687 A1
(43) Pub. Date: Dec. 15, 2022

(54) System and Method for Sensory Transmission Block by Electrical Stimulation of Neural Tissue

(71) Applicant: University of Connecticut, Farmington, CT (US)

(72) Inventors: Bin Feng, Storrs, CT (US); Longtu Chen, Coventry, CT (US); Phillip P. Smith, Storrs, CT (US)

(73) Assignee: University of Connecticut, Farmington, CT (US)

(21) Appl. No.: 17/840,869

(22) Filed: Jun. 15, 2022

Related U.S. Application Data
(60) Provisional application No. 63/210,671, filed on Jun. 15, 2021.

(51) Int. Cl. A61N 1/36; A61N 1/05

(52) U.S. Cl. CPC A61N 1/36071; A61N 1/0551; A61N 1/36057; A61N 1/36139; A61N 1/36171

(57) ABSTRACT

Systems and methods are provided for electrical stimulation using electrical neurostimulators to treat neurological disorders. In exemplary implementations, the systems and methods effectuate selective sensory transmission block by spatially and temporally patterned multichannel, closed-loop electrical stimulation of neural tissue across the intervertebral foramina. The systems and methods optimize stimulation parameters according to the conduction velocity of afferents leading to effective neural transmission block. The systems and methods provide a dramatic improvement of selective transmission block, e.g., in a sub-population of unmyelinated C-fibers and slow-conducting Aδ fibers, by combining spatial, frequency and temporal parameters in the disclosed stimulation paradigm. An optimized start and termination of the stimulation may be implemented, as desired, thereby reducing overall energy consumption by reducing the stimulus strength during the maintenance phase of neural transmission block.

### CROSS-REFERENCE TO RELATED APPLICATION

The present application claims priority benefit to a U.S. provisional patent application entitled “System and Method for Selective Sensory Transmission Block by Spatially and Temporally Patterned Multichannel, Closed-Loop Electrical Stimulation of Neural Tissue Across the Intervertebral Foramina,” which was filed on Jun. 15, 2021, and assigned Ser. No. 63/210,671. The entire content of the foregoing provisional application is incorporated herein by reference.

### STATEMENT OF GOVERNMENT SUPPORT

This invention was made with Government support under Grant Nos. DK114546 and DK120824 awarded by the National Institutes of Health. The Government has certain rights in the invention.

### FIELD OF THE DISCLOSURE

The invention described herein is directed to systems and methods for electrical stimulation using electrical neurostimulators to treat neurological disorders. In exemplary implementations, the disclosed systems and methods effectuate selective sensory transmission block by spatially and temporally patterned multichannel, closed-loop electrical stimulation of neural tissue across the intervertebral foramina. The disclosed systems and methods offer an advantageous electrical stimulation strategy and modality to treat neurological disorders, including specifically chronic pain.

### BACKGROUND

Visceral pain is the leading symptom of functional gastrointestinal disorders like irritable bowel syndrome (IBS) that affects up to 20% of the U.S. population. Pharmacological management of visceral pain in IBS remains challenging as evidenced by many failed clinical trials of drugs to modify pain end points. Neuromodulation has emerged as a non-drug alternative for managing many types of chronic pain. For example, electrical stimulation of spinal cord (spinal cord stimulation [SCS]) and peripheral nerves have been widely reported to manage low back pain, neuropathic pain, and complex regional pain syndrome. In contrast, neuromodulation to manage visceral pain is much less frequent in clinics and rarely reported in the literature despite the widespread incidence of visceral pains.

Currently, it is routine for the parameters of neuromodulation in the treatment of pain to be arbitrarily determined leading to unsatisfactory pain inhibition effect. Current neuromodulation approaches do not offer selectivity of stimulated tissues, leading to undesired off-target side effects, such as excessive area of paresthesia and disturbance of normal sensory and motor functions. Moreover, existing approaches to neuromodulation are highly inefficient from an energy consumption standpoint.

Conventional stimulation relies on activating fast-conducting myelinated A-fibers to treat pain which follows the “Gate Control Theory”. For pain originating from organs that lack prominent Aα- and Aβ-fiber innervations, conventional neuromodulation is not efficient in suppressing the pain. Traditional neurostimulators generally treat chronic pain in patients by implementing a “Gate Control Theory.” Neuromodulation techniques such as spinal cord stimulation (SNS), peripheral nerve stimulation (PNS) and dorsal root ganglion (DRG) stimulation are used to manage pain by activating low-threshold sensory afferents to elicit pain inhibition effects according to “Gate Control Theory” first proposed by Melzack et al.

However, the “Gate Control Theory” requires that the origin of the pain be from organs or tissues with prominent innervation of myelinated sensory afferents. Pain from visceral organs that lack innervations by myelinated afferents cannot be efficiently treated using the existing neuromodulation strategies. Existing technologies are generally not effective in treating pain from organs that lack a prominent innervation component of fast-conducting myelinated A-fibers, including, intestine, pelvic floor organs, prostate, bladder, and urethra.

Alternatively, local injection of chemical anesthesia has been used as an expedient to temporarily alleviate visceral pain by blocking afferent neural transmission.

Despite efforts to date, a need remains for electrical stimulation-based systems and methods that can effectively treat neurological disorders, including specifically chronic pain. These and other objectives are satisfied by the systems and methods disclosed herein.

### SUMMARY

The present disclosure provides advantageous systems and methods for electrical stimulation using electrical neurostimulators to treat neurological disorders. In exemplary implementations, the disclosed systems and methods effectuate selective sensory transmission block by spatially and temporally patterned multichannel, closed-loop electrical stimulation of neural tissue across the intervertebral foramina. The disclosed systems and methods optimize stimulation parameters according to the conduction velocity of afferents leading to effective neural transmission block. The disclosed systems and methods provide a dramatic improvement of selective transmission block, e.g., in a sub-population of unmyelinated C-fibers and slow-conducting Aδ fibers, by combining spatial, frequency and temporal parameters in the disclosed stimulation paradigm. According to exemplary implementations of the present disclosure, an optimized start and termination of the stimulation is implemented, as desired, thereby reducing overall energy consumption by reducing the stimulus strength during the maintenance phase of neural transmission block.

An algorithm is described herein for automatically initiating neuromodulation to block the abnormal neural activity and adjusting the stimulus intensity and frequency after transmission block is achieved, thereby significantly improving efficiency of the neuromodulation regimen.

The disclosed systems/methods may be used to block slow-conducting C-fibers (unmyelinated) and Aδ-fibers (thinly myelinated) to stop nociceptive signals from transmitting to the spinal cord, thereby facilitating treatment of pain arising from visceral organs that lack fast-conducting A fibers.

The disclosed systems and methods thus offer an advantageous electrical stimulation strategy and modality to treat neurological disorders, including specifically chronic pain. Additonal features and functions of the disclosed systems and methods will be apparent from the detailed description which follows, particularly when read in conjunction with the accompanying figures.

### BRIEF DESCRIPTION OF THE FIGURES

To assist those of skill in the art in making and using the systems and methods of the present disclosure, reference is made to the accompanying figures, wherein:

FIG. 1A is a plot of optimal blocking frequency (OBF) and conduction velocity (CV) for afferents;

FIG. 1B is a plot showing that neuromodultation frequencies above or below the OBF block a much lower proportion of afferents;

FIG. 1C is a schematic depiction of selective block of a sub-population of C-fibers and slow-conducting Aδ fibers that receive combined stimulation at their T-junction;

FIG. 1D is a schematic depiction of selective afferent block by spatial and temporal summation of low-frequency stimulation at multiple sites to stimulate with OBF at focal afferent population;

FIG. 2 is an exemplary flowchart for algorithmic delivery of stimulation by monitoring ectopic afferent activities from the dorsal root, closed-loop multichannel neural stimulation, and intelligent termination of stimulus or attenuation of stimulus intensity and frequency to maintain sensory block at a low rate of electrical power consumption;

FIG. 3 is a schematic cross-sectional view of a nerve showing subthreshold stimuli from multiple stimulating electrodes that interfere with each other to form suprathreshold stimuli at the desired location to stimulate a specific afferent;

FIG. 4A is a schematic depiction of intervertebral foramina anatomy;

FIG. 4B is a schematic depiction of an exemplary system according to the present disclosure deployed relative to intervertebral foramina anatomy;

FIG. 5A is a schematic side view of electrode arrays targeting inner axons and superficial axons;

FIG. 5B is a schematic top view of the electrode arrays depicted in FIG. 5A;

FIG. 5C is a schematic cross-sectional view of the electrode arrays of FIGS. 5A and 5B targeting the dorsal root and spinal nerve;

FIG. 6 is a schematic depiction of two stimulating electrode arrays targeting DRG and T-junction;

FIG. 7A is a schematic depiction of an experimental test regimen in which single-fiber recordings are obtained from individual different axons according to the present disclosure;

FIG. 7B is a representative image frame of recorded GCaMP6f transients generated in an experimental study according to the present disclosure;

FIG. 7C is a plot of summarized chronaxies of DRG simulation determined by single-fiber recordings in naïve mice (solid dots) and GCaMP6f recordings in naive and TNBS-treated mice (hollow dots); asterisks indicate P<0.05. (DRG—dorsal root ganglion; Med, medium-diameter neuron; Sml, small diameter neuron; TNBS, 2,4,6-trinitrobenzenesulfonic acid);

FIGS. 8A and 8B are images showing dissection of colorectum-PN-DRG-DR according to an experimental study of the present disclosure;

FIG. 8C is a schematic depiction of an experimental set-up according to the present disclosure;

FIG. 8D is a schematic of a protocol to assess the efficacy of DRG stimulation on colorectal afferent neural transmission. Action potentials evoked by CRD are recorded before (control), immediately after, and 15 to 30 minutes after (recovery) DRG stimulation. The duration of pressure steps in the CRD protocol for TNBS-treated and naive mice is 5 and 10 seconds, respectively, the interval between successive pressure steps is 8 seconds. (CRD, colorectal distension, DR, dorsal root; DRG, dorsal root ganglion; PN, pelvic nerve; TNBS, 2,4,6-trinitrobenzenesulfonic acid);

FIG. 9A is a schematic depiction of the harvesting of the SN-DRG-DR according to an experimental study of the present disclosure;

FIGS. 9B and 9C are schematic depictions of temporally synchronized SN and DRG stimulation protocols according to the present disclosure;

FIG. 10A is an image of the dorsal side of the L6 vertebral segment as dissected to expose muscle layers in an experimental study according to the present disclosure;

FIG. 10B is an image of DRG stimulating electrode fixation in an experimental study according to the present disclosure;

FIG. 10C is a magnified view of the exposed L6 DRG and stimulating electrode in an experimental study according to the present disclosure;

FIG. 10D is a schematic depiction of a setup for recording the EMG responses to CRD and conducting L6 DRG stimulation according to an experimental study according to the present disclosure;

FIGS. 11A and 11B are plots using a colorectum-PN-DRG-DR preparation that show inhibition of colorectal afferent transmission by suprathreshold DRG stimulation in both naïve (FIG. 11A) and TNBS-treated mice (FIG. 11B);

FIG. 11C is a magnified view of the dashed box in FIG. 11B showing single-unit action potentials in an experimental study according to the present disclosure;

FIGS. 12A and 12B are plots showing frequency-dependent inhibition of colorectal afferent transmission by suprathreshold DRG stimulation in both naïve (FIG. 12A) and TNBS-treated (FIG. 12B) mice in an experimental study according to the present disclosure;

FIG. 13A are single-fiber recordings from typical Aδ and C fibers in an experimental study according to the present disclosure;

FIG. 13B are plots showing CD of a typical Aδ fiber and a C fiber plotted against the SN stimulation index in an experimental study according to the present disclosure;

FIGS. 14A-14D are plots showing the neuromodulatory effects of suprathreshold DRG stimulation in an experimental study according to the present disclosure;

FIG. 15A is a histogram of baseline CV from Aδ fibers and C fibers subjected to DRG stimulation in an experimental study according to the present disclosure;

FIG. 15B is a scatter plot of the afferents shown in FIG. 15A at different CV undergoing DRG stimulation for tested frequencies in an experimental study according to the present disclosure;

FIG. 16A shows representative electromyography (EMG) activities recorded at the abdominal oblique musculature during the CRD protocol in an experimental study according to the present disclosure;

FIG. 16B provides plots in which normalized area under the curve (AUC) values of EMG activity in response to CRD is shown in an experimental study according to the present disclosure (EMG responses to CRD before DRG stimulation serve as a control, which are also recorded immediately after (After) and 15 to 30 minutes after the termination of DRG stimulation (Recovery)); and

FIG. 16C provides normalized AUC values of EMG activities in response to 60 mm Hg pressure distension immediately after DRG stimulation at 10, 50 and 100 Hz in an experimental study according to the present disclosure.

### DESCRIPTION OF EXEMPLARY EMBODIMENTS

The present disclosure provides an advantageous neural stimulation system and method to suppress pain sensation by low-frequency (e.g., less than about 5 Hz) electrical stimulation, which achieves selective, closed-loop, intelligent and reversible transmission block in sensory neurons, i.e., the afferents.

The disclosed electrical stimulation includes a stimulating electrode that is used to target the segment of afferents within the intervertebral foraminal canal, i.e., the dorsal root, dorsal root ganglion (DRG), T-junction and spinal nerve. According to the present disclosure, each of the neuromodulatory targets can be stimulated individually or simultaneously with one or more other targets. A recording electrode is positioned/implanted at the dorsal root to sense and record afferent neural activities. The sensed afferent neural activities drive closed-loop, intelligent control of a neural stimulation algorithm associated with the disclosed system, the neural stimulation algorithm adapted to be run by a processing unit. Coupled with the stimulating and recording electrodes, the neural stimulation algorithm functions to achieve effective, selective, and energy-efficient afferent neuromodulation to suppress pain.

The present disclosure offers significant benefits relative to conventional neurostimulation systems and methods, including the following.

First, the systems and methods of the present disclosure provide/deliver a direct sensory transmission block through optimized electrical stimulation. This mechanism of action is different from conventional approaches that evoke paresthesia to block pain in spinal cord stimulation or peripheral nerve stimulation.

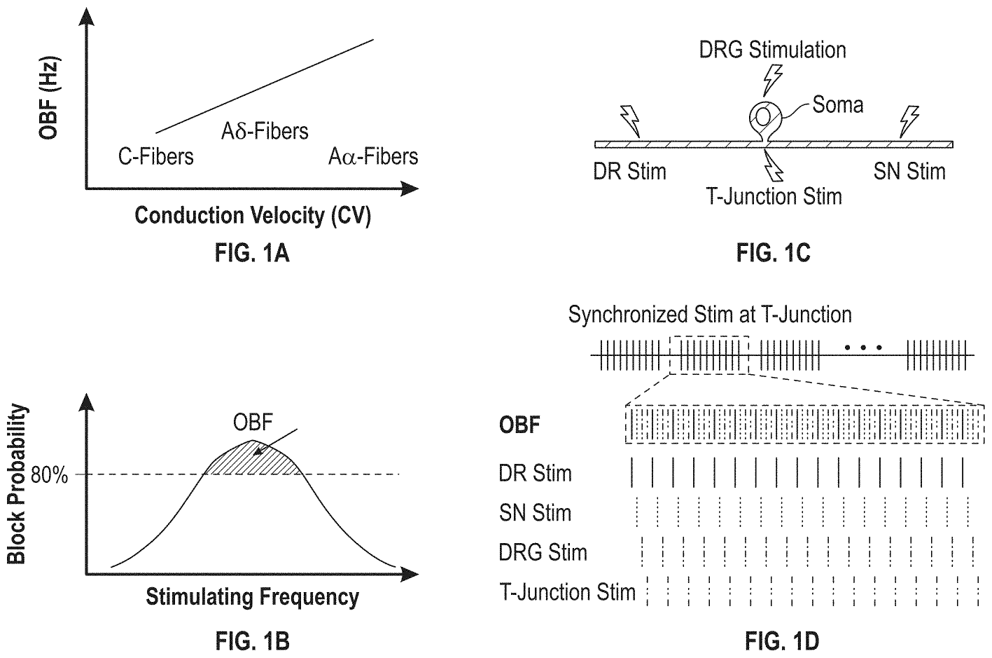

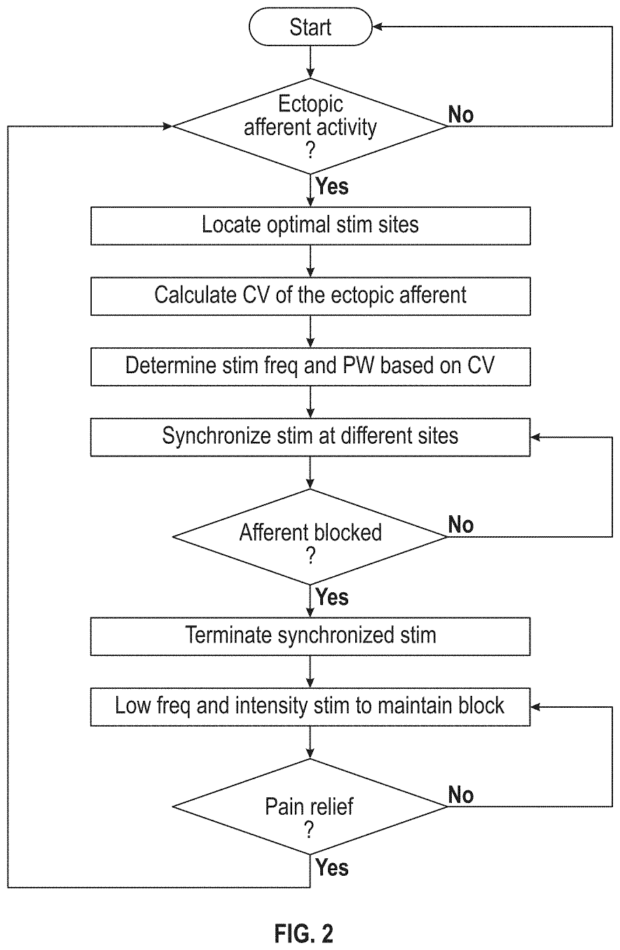

Second, the systems and methods of the present disclosure provide/deliver selective transmission block of unmyelinated C-fibers and slow-conducting Aδ-fibers, the sensitization of which plays critical roles in chronic pain. This advantageous functionality is achieved according to the present disclosure based on structural/functional design features that lead to highly selective block.
First, optimal neuromodulation frequency of specific afferents is determined according to the present disclosure based on the conduction velocity (CV) of that afferent. In particular, based on experimental data, an approximately proportional relationship has been identified between the optimal blocking frequency (OBF) and CV for afferents, as shown in FIG. 1A. Frequencies above or below the OBF block a much lower proportion of afferents, as demonstrated in FIG. 1B.
Second, the temporal and spatial summation of multichannel stimulation allow selective block of a sub-population of C-fibers and slow-conducting Aδ fibers that receive combined stimulation at their T-junction from multiple electrode leads, each of which delivers stimuli at frequencies much lower than the OBF (typically <5 Hz), as displayed in FIG. 1C and FIG. 1D. More specifically, FIGS. 1A-1D demonstrate that an optimal blocking frequency (OBF) is dependent upon the conduction velocities (CV) of each afferent. As shown in FIG. 1A, the OBF is approximately proportional to the CV and has the highest rate of conduction block for that particular afferent, as shown in FIG. 1B. Selective afferent block is achieved by spatial and temporal summation of low-frequency stimulation at multiple sites, as shown in FIG. 1C, leading to stimulation with OBF at focal afferent population, as shown in FIG. 1D.
Third, the systems and methods of the present disclosure provide/deliver smart/optimized stimulation by monitoring ectopic afferent activities from the dorsal root, closed-loop multichannel neural stimulation, and intelligent termination of stimulus or attenuation of stimulus intensity and frequency to maintain sensory block at a low rate of electrical power consumption. The disclosed neural stimulators also record afferent activities and determine their fiber types (e.g., C-, Aδ-, Aβ-, or Aα-fibers) based upon conduction velocity (CV). An exemplary program flowchart is shown in FIG. 2. Specifically, FIG. 2 depicts a schematic of an exemplary smart/optimized stimulation algorithm for pain relief according to the present disclosure.

The disclosed systems and methods may be implemented in various ways, e.g., by medical device manufacturing companies that incorporate the disclosed electrical stimulation strategy into neurostimulators systems/hardware. For example, existing neurostimulators can be further developed to incorporate the stimulus parameters described herein including, but not limited to, neurostimulators targeting peripheral nerve, spinal cord nerve roots, DRG and T-junction.

The disclosed systems/methods offer significant benefits to patients with various pain symptoms, such as limb pain, neuropathic pain, visceral pain, etc. The ability to manage pain with the disclosed neuromodulation techniques using/adopting a low-frequency, patterned stimulation protocol coupled with the disclosed electrode array and by effecting selective block of pain sensation while mitigating the impact on normal sensory function.

Of note, the disclosed low-frequency (typically <5 Hz) electrical stimulation method does not suppress pain by activating fast-conducting myelinated A-fibers, but by direct nerve block through stimulation of the neural tissue inside the foraminal canal. Compared with existing neurostimulators, the neuromodulation approach described in the present disclosure is superior in managing pain from organs and tissues that lack prominent A-fiber innervations, especially for managing visceral pain.

The disclosed systems and methods advantageously achieve pain relief without causing paresthesia, a non-painful tingling sensation resulting from activation of low-threshold myelinated

A-fibers. Conventional neuromodulation requires the sensation of paresthesia to mask the sensation of pain from the same region. Some patients are not bothered by the constant sensation of paresthesia, but some report reduced sleep quality and difficulty to focus due to the presence of paresthesia. The paresthesia-free neuromodulation of the disclosed system/method offers benefits and is attractive to a larger patient population.

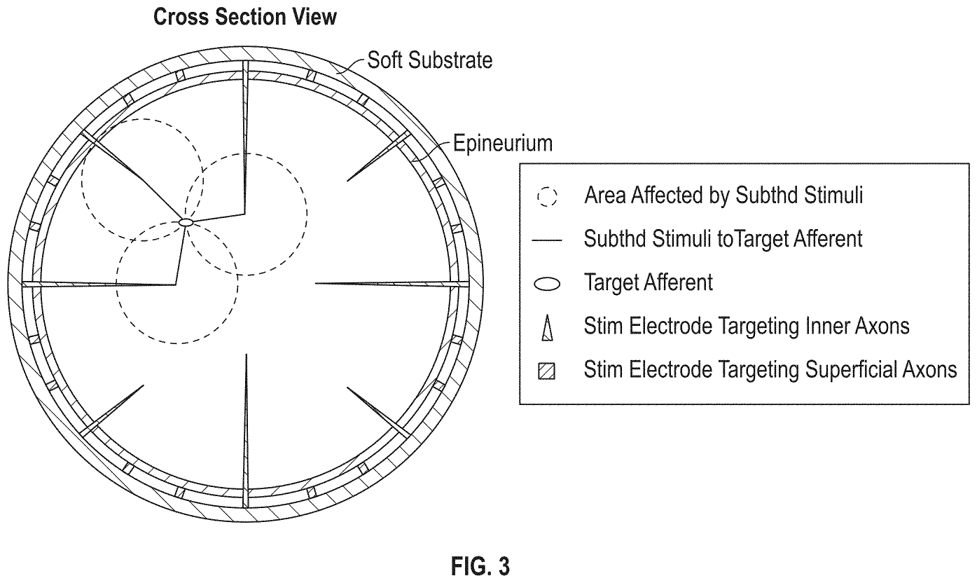

The disclosed systems and methods achieve dramatically increased selectivity of electrical neuromodulation, e.g., blocking only a sub-population of unmyelinated C-fibers and slow-conducting As-fibers. Yet, the blocked afferents do not need to be in close proximity to the stimulating electrode leads. Subthreshold stimuli from multiple stimulating electrodes interfere with each other to form suprathreshold stimuli at the desired location to stimulate the specific afferent, as shown in FIG. 3. This is in stark contrast to the conventional spinal cord stimulation, peripheral nerve stimulation or DRG stimulation in which large Aα- and Aβ-fibers close to the electrode leads are readily evoked whereas unmyelinated C-fibers and thinly myelinated Aδ fibers are generally unaffected.

The increased selectivity of the present disclosure is achieved by spatial and temporal summation of synchronized low-frequency stimulation (typically <5 Hz) at multiple neural tissue in the foramen. From the spatial and temporal summation, focal regions of the targeted afferent will be stimulated at optimal blocking frequency (OBF) for efficient transmission block (see, e.g., FIGS. 1A-1D). This beneficial effect is confirmed by experimental results showing that the frequency of electrical stimulation determines the likelihood of afferent transmission block:
10-50 Hz stimulation blocks most unmyelinated C-fibers and slow-conducting Aδ fibers while leaving fast-conducting Aα- and Aβ-fibers unaffected; Frequency below 5 Hz does not generally block afferent transmission (absent spatial and temporal summation of synchronized low-frequency stimulation as disclosed herein); and
Frequency over 500 Hz does not block C- or Aδ-fibers, but potentially affects fast-conducting Aα- and Aβ-fibers.

As noted above, FIG. 3 depicts a schematic of spatial summation of sub-threshold stimulation to achieve suprathreshold stimulation at the focal region.

The described neural stimulator creatively determines the stimulus pulse width and optimal blocking frequency (OBF) based on the measured afferent conduction velocity (CV), which objectively categorized afferents into C-, Aδ-, Aβ- and Aα-fiber subtypes. The stimulation parameters are then tailored to fit each individual afferent leading to effective neural transmission block.

The systems and methods of the present disclosure significantly attenuate off-target side effects by increasing selectivity in the spatial and frequency domain. In the spatial domain, a multichannel stimulating electrode array is used inside the foraminal canal to allow localized stimulation of dorsal root, DRG, T-junction and spinal nerve. The multichannel recording electrode array implanted to the dorsal root is capable of recording neural activity from localized axons. The algorithm compares the recorded pain-related neural activity with the neural response evoked by stimulation and then accurately locates the relevant stimulating electrodes to achieve spatial selectivity. Also, under the circumstance that the targeted afferent locates far from any single stimulating electrode, multiple subthreshold stimuli from the surrounding electrodes will be delivered to form suprathreshold stimuli at the focal region, as shown in FIG. 3, which provides additional selectivity of stimulation to mitigate off-target effects. In the frequency domain, either stimulate individual sites in the foraminal canal (dorsal root, DRG, T-junction and spinal nerve) at the optimal blocking frequency (OBF) determined by CV or synchronize stimulation at different sites with lower frequency to reach a combined stimulation frequency equal to the OBF. By this means, the described algorithm can block specific afferents while spare the others to achieve selective neuromodulation.

The disclosed systems/methods can automatically detect aberrant pattern of neural activity and immediately initiates neuromodulation to block the transmission by implementing a recording electrode array at the dorsal root. During the process of stimulation, the algorithm removes artifact engendered by electrical stimulation and thus can monitor the neural response to stimulation in real time. The algorithm will terminate the delivery of stimuli after detecting transmission block and will continue to monitor if the ectopic transmission relapses, under which circumstance the algorithm will provide stimulation at a lower frequency and intensity to maintain the blocking effect.

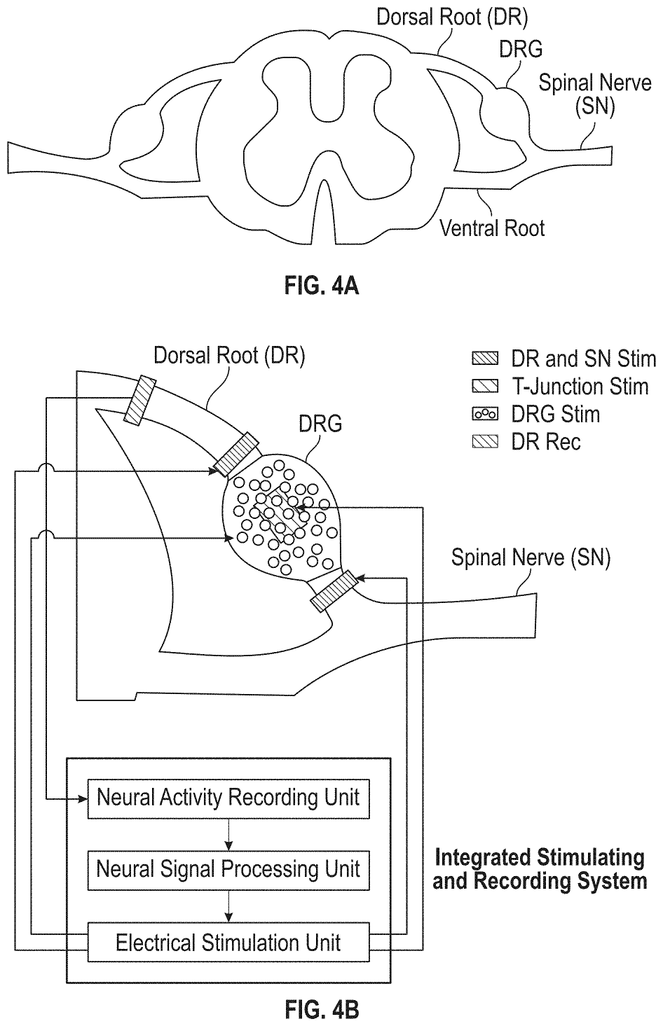

In an exemplary embodiment, hardware associated with the disclosed systems/methods include a plurality of stimulating electrode arrays, e.g., four (4) stimulating electrode arrays, one recording electrode array, and an integrated stimulating and recording system for regulating the neuromodulation process, as schematically depicted in FIG. 4B as deployed relative to the intervertebral foramina anatomy of FIG. 4A. Thus, FIG. 4B depicts an exemplary neural stimulating system according to the present disclosure that targets the neural tissue across the intervertebral foramina, including the dorsal root, DRG, T-junction, and spinal nerve.

In the exemplary embodiment/implementation depicted in FIG. 4B, the four stimulating electrode arrays target four different regions of neuronal tissues inside the foramen: i.e., the dorsal root, spinal nerve, DRG, and T-junction.

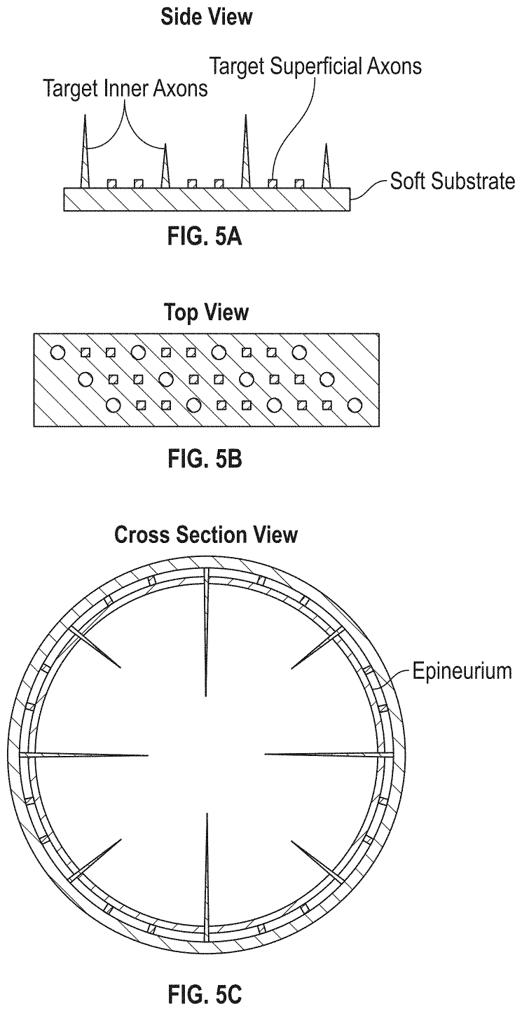

With reference to FIGS. 5A-5C, schematic depictions of the layout of two electrode arrays targeting the dorsal root and spinal nerve are provided, wherein the electrode arrays include both surface and penetrating electrodes. The superficial electrodes can be fabricated in various geometries, e.g., rectangular, square, round and/or oval shapes. The sizes of the electrodes can be adjusted to accommodate different application scenarios. The embedded electrodes are fabricated using flexible materials, such as carbon fiber, to counteract potential relative movement between the electrode and nerve tissue. In some applications, only the superficial electrodes are used to stimulate the dorsal root and spinal nerve, while in other implementations, only the embedded electrodes are used.

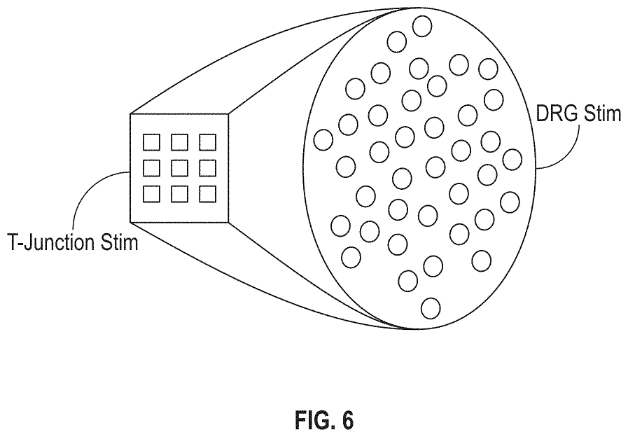

FIG. 6 provides a further schematic depiction of two stimulating electrode arrays targeting DRG and T-junction, the electrode arrays only including surface electrodes mounted relative to a flexible substrate.

In exemplary implementations, a recording electrode array is implanted at the dorsal root to directly monitor afferent neural activities transmitting towards the spinal cord. As shown in FIG. 5, the recording electrode array generally consists of superficial electrodes and embedded electrodes to record from superficial and deeper axons, respectively. The embedded electrodes penetrating the dorsal root are typically fabricated with flexible materials, such as carbon fiber, to allow/facilitate a stable nerve-electrode interface. The substrate of the recording electrode is flexible and, in exemplary embodiments, the substrate may be bent over to cover the whole (or substantially the whole) surface of the dorsal root. The layout of superficial electrodes and embedded electrodes on the substrate can be customized according to the application.

As shown in FIG. 4B, the integrated stimulating and recording system of the present disclosure generally consists of a neural activity recording unit, a neural signal processing unit and an electrical stimulation unit. The neural activity recording unit amplifies, filters and digitizes the neural signal from the recording electrode array. The neural signal processing unit analyzes the recorded neural signal to detect ectopic neural activity and determine an optimal neuromodulation strategy for selective neural transmission block. After receiving the command from the neural signal processing unit, the electrical stimulation unit generates and delivers stimuli to selectively block a subpopulation of unmyelinated C-fibers and slow conducting Aδ fibers by the spatial and temporal summation of low-frequency stimulation (typically <5 Hz) from multiple electrodes. In the meanwhile, the neural signal processing unit monitors the neuromodulation effects in real time to re-analyze the relevant data and adjust the stimulation protocol to achieve closed-loop neuromodulation.

In an exemplary embodiment, to achieve optimized and selective afferent transmission block, the disclosed algorithm embedded in the neural signal processing unit possesses a host of unique and advantageous features. An exemplary program flowchart is shown in FIG. 2. According to exemplary embodiments, the disclosed algorithm is adapted to provide the following features and functions:

1) The algorithm automatically detects ectopic neural activity from the afferents. It extracts firing rate and morphological parameters to distinguish ectopic neural activity from normal sensory transmission.

2) The algorithm supports spatial selectivity of modulation by locating the optimal site(s) for stimulation. The subpopulation of afferents responsible for ectopic activities recorded from dorsal roots are located by electrical stimulation from the electrode arrays targeting the dorsal root, DRG, T-junction and spinal nerve. The stimulating electrode closest to the ectopically firing afferent is generally used to selectively block neural transmission. Under the circumstance that the targeted afferent locates far from any single stimulating electrode, multiple subthreshold stimuli from the surrounding electrodes may be delivered to form suprathreshold stimuli at the targeted regions, as shown in FIG. 3, which will further increase selectivity and mitigate off-target effects. In an exemplary embodiment, to allow recording of electrically evoked action potentials from the dorsal root, an electrical artefact removal algorithm disclosed in the literature may be implemented. (L Chen, Frontiers in Neuroscience, 2020, doi.org/10.3389/fnins.2020.00709).

3) The algorithm optimizes the stimulus pulse width and frequency for the targeted afferent axons according to the measured conduction velocity, which objectively determines their fiber types (C-, Aδ-, Aβ-, or Aα-fibers).

4) The algorithm may be adapted to achieve temporal selectivity of modulation by synchronized low-frequency (typically <5Hz) stimulation at multi-sites inside the foraminal canal, as illustrated in FIG. 1C and FIG. 1D. Afferents with different conduction velocities (i.e., C-, Aδ-, Aβ-, and Aα-fibers) are generally susceptible to transmission block by different range(s) of stimulation frequencies. Low-frequency (<5 Hz) stimulation generally does not block afferent transmission whereas 10-50 Hz stimulation selectively blocks most unmyelinated C-fibers and slow conducting Aδ-fibers while sparing fast conducting Aα- and Aβ-fibers. By synchronizing the low-frequency (<5 Hz) stimulation from multiple sites, focal regions with additive optimal blocking frequency advantageously deliver selective transmission block of most unmyelinated C-fibers and slow conducting Aδ-fibers.

5) The algorithm allows closed-loop control to optimize the stimulation protocol. By using the aforementioned artefact removal algorithm (L Chen, Frontiers in Neuroscience, 2020, doi.org/10.3389/fnins.2020.00709), the disclosed system/method allows continuous monitoring and detection of ectopic sensory afferent activities at the dorsal root, even during the process of electrical stimulation. The algorithm is generally adapted to automatically terminate the stimulation or reduce the stimulus frequency and intensity after detecting successful transmission block by the stimulation. To maintain transmission block, stimulus frequency and intensity can be reduced compared to those used to achieve transmission block.

Blocking Peripheral Drive from Colorectal Afferents by Subkilohertz Dorsal Root Ganglion Stimulation

Clinical evidence indicates dorsal root ganglion (DRG) stimulation effectively reduces pain without the need to evoke paresthesia. This paresthesia-free anesthesia by DRG stimulation can be promising to treat pain from the viscera, where paresthesia usually cannot be produced. Mechanisms and parameters for DRG stimulation using an ex vivo preparation with mouse distal colon and rectum (colorectum), pelvic nerve, L6 DRG, and dorsal root in continuity are described herein. Single-fiber recordings from split dorsal root filaments were conducted and the effect of DRG stimulation on afferent neural transmission assessed. Based on the foregoing assessment, the optimal stimulus pulse width was determined by measuring the chronaxies of DRG stimulation to be below 216 μLs. Of note, the data indicates spike initiation likely at attached axons rather than somata. Subkilohertz DRG stimulation significantly attenuates colorectal afferent transmission (10, 50,100, 500, and 1000 Hz), of which 50 and 100 Hz show superior blocking effects. Synchronized spinal nerve and DRG stimulation reveals a progressive increase in conduction delay by DRG stimulation, suggesting activity-dependent slowing in blocked fibers. Afferents blocked by DRG stimulation show a greater increase in conduction delay than the unblocked counterparts. Midrange frequencies (50-500 Hz) are more efficient at blocking transmission than lower or higher frequencies. In addition, DRG stimulation at 50 and 100 Hz significantly attenuates in vivo visceromotor responses to noxious colorectal balloon distension. This reversible conduction block in C-type and Aδ-type afferents by subkilohertz DRG stimulation likely underlies the paresthesia-free anesthesia by DRG stimulation, thereby enhancing chronic visceral pain management.

1. Methods

All experimental procedures were approved by the University of Connecticut Institutional Animal Care and Use Committee. All the mice used in the following experiments were housed in pathogen-free facilities which are Public Health Service assured and American Association for Accreditation of Laboratory Animal Care accredited following the Guide for the Care and Use of Laboratory Animals Eighth Edition. Mice resided in individual ventilated caging systems in polycarbonate cages (Animal Care System M.I.C.E.) and were provided with contact bedding (Envigo T7990 B.G. Irradiated Teklad Sani-Chips). Mice were fed ad lib with either 2918 Irradiated Teklad Global 18% Rodent Diet or 7904 Irradiated 52335 Mouse Breeder Diet supplied by Envigo and supplied with reverse osmosis water chlorinated to 2 ppm using a water bottle. Nestlets and huts were supplied for enrichment. Rodent housing temperature was set for 73.5° F. with a range from 70 to 77° F. Humidity was set for 50% with a range of 35% to 65%. Mice were housed with a maximum of 5 animals per cage. All animals were housed on a 12:12 light: dark cycle. Animals were observed daily by the animal care services staff. Cages were changed every 2 weeks.

a. Intracolonic 2,4,6-trinitrobenzenesulfonic Acid Treatment

Mice were anesthetized by isoflurane inhalation, transanally administered with TNBS (0.2 mL at 10 mg/mL in 50% ethanol; Sigma-Aldrich, St. Louis, Mo.) using a 22-gauge feeding needle (#18061-22; Fine Science Tools, Foster City, Calif.), and held in a head down position (˜30°) for 5 minutes to preserve TNBS in the colorectum. Dietary gel (NGB-1; Bio-Serv, Flemington, N.J.) was provided to mice showing severe weight loss (>5% original body weight). Mice at 7 to 14 days after TNBS treatment were used in current study, a time span of colorectal hypersensitivity as systematically characterized in a previous study. [See, Feng B, La J H, Tanaka T, Schwartz E S, McMurray T P, Gebhart G F. Altered colorectal afferent function associated with TNBS-induced visceral hypersensitivity in mice. Am J Physiol Gastrointest Liver Physiol 2012; 303:G817-24.]

b. Characterization of the Strength-Duration Curve of Dorsal Root Ganglion Stimulation

To achieve efficient electrical stimulation, it is necessary to deliver a stimulus with pulse width close to the chronaxie of the strength-duration curve at which excitable tissues are activated by the least amount of electrical energy. The strength-duration curve of mouse DRG stimulation was determined by measuring the threshold stimulus amplitude at different stimulus pulse widths from 20 μs to 2 ms. To accurately assess the stimulus threshold, the evoked DRG responses were recorded using 2 independent methods: single-fiber recordings at the attached DRs from C57BL/6 mice and optical recordings of intracellular Ca2+ transients in DRG somata from a transgenic mouse strain expressing GCaMP6f in sensory neurons driven by the VGLUT2 promotor.

i. Single-Fiber Recordings of Evoked Action Potentials

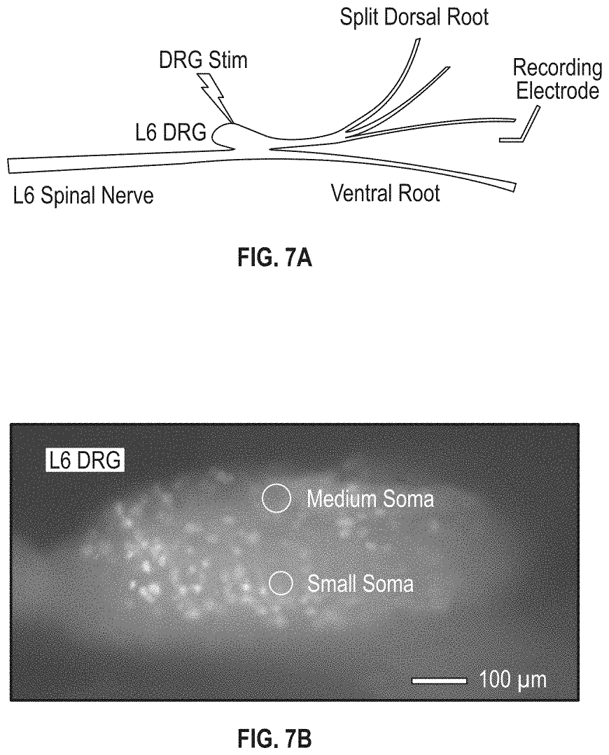

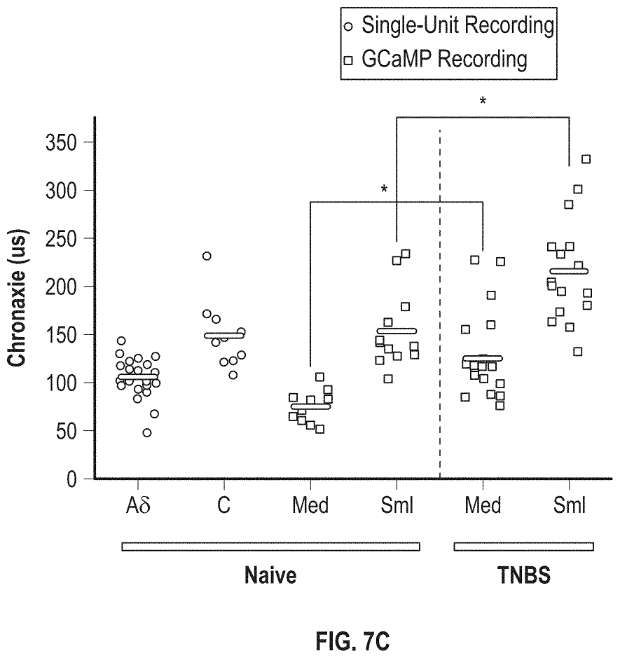

C57BL/6 mice of both sexes (8-16 weeks, 25-35 g, N=10) were anesthetized by 2% isoflurane inhalation followed by intraperitoneal and intramuscular injection of a ketamine/xylazine cocktail (100/10 mg per kg weight), and then euthanized by exsanguina-tion from the right atrium and transcardiac perfusion from the left ventricle with oxygenated (95% O₂, 5% CO₂) ice-cold Krebs solution containing (mM) 117.9 NaCl, 4.7 KCl, 25 NaHCO₃, 1.3 NaH₂PO₄, 1.2 MgSO₄, 2.5 CaCl₂, and 11.1 D-glucose. Dorsal pediculectomy was performed to expose the spinal cord and DRG from T12 to S1 segments. Exsanguinated mouse carcass was then placed in a dissection chamber circulated with oxygenated ice-cold Krebs solution. The L6 DRG with an attached DR and a segment of the spinal nerve (SN) was carefully harvested in continuity and transferred to a custom-built re-cording chamber consisting of a tissue chamber and an adjacent recording chamber. The L6 DRG was placed in the tissue chamber perfused with oxygenated Krebs solution at 30° C., and the DR was gently pulled into the recording chamber filled with mineral oil (Fisher Scientific, East Greenwich, R.I.) to enhance the signal-to-noise ratio of the recording. The L6 DR was carefully split into fine filaments (˜10 μm) to achieve single-fiber recordings from individual afferent axons as shown in FIG. 7A based on a protocol set forth in the following publication: Chen L, Ilham S J, Guo T, Emadi S, Feng B. In vitro multichannel single-unit recordings of action potentials from mouse sciatic nerve. Biomed Phys Eng Express 2017; 3:045020.

ii. Optical GCaMP6f Recordings of Evoked Action Potential

Ai95 mice (C57BL/6 background) possessing homozygous GCaMP6f (strain #28865; Jackson Laboratory, CT) were cross-bred with mice carrying homozygous VGLUT2-Cre (strain #28863; Jackson Laboratory, CT) to express GCaMP6f in glutamatergic neurons expressing type 2 vesicular glutamate transporter (VGLUT2), which is extensively present in sensory neurons innervating the colorectum. Naive (N=7) and TNBS-treated (N=8) transgenic mice of both sexes (8-16 weeks and 25-35 g) with heterozygous GCaMP6f and VGLUT2-Cre genes (i.e., VGLUT2/ GCaMP6f) were used for optical electrophysiological recordings. The L6 DRG was harvested following the same procedure for single-fiber recordings described above. To conduct optical GCaMP6f recordings, an upright fluorescent microscope (BX51WI; Olympus, Waltham, Mass.) coupled with a water immersion 10× objective (UMPLFLN 10XW, 0.3 NA) was used, which allowed visualization of the whole L6 DRG with sufficient resolution to detect Ca2+ transients in individual DRG somata. The DRG was illuminated with a halogen epi-illumination light source and video recorded the evoked GCaMPf6 transients at 1920×1080 pixel resolution (2×2 bins) and 60 to 100 frames per second using a high-speed ultra-low noise scientific complementary metal-oxide-semiconductor camera (Zyla-4.2P, 82% quantum efficiency; Andor Technology, South Windsor, Conn.). Displayed in FIG. 7B is a representative image frame of recorded GCaMP6f transients. The DRG diameters were measured post hoc on captured images in ImageJ (NIH, Bethesda, Md.). The actual size was derived by converting the pixel size to microns (2 μm/pixel).

iii. Dorsal Root Ganglion Stimulation Protocols to Characterize the Strength-Duration Curve

To electrically stimulate the DRG, a blunt-tipped needle electrode (FHC, platinum-iridium, tip size 1-25 μm) placed in contact with, but not penetrating, the capsule of the L6 DRG (i.e., the continuation of the dura mater enclosing the spinal cord) was used to deliver constant current stimulation (701C stimulating module; Aurora Scientific Inc, Aurora, Canada). Charge-balanced bipolar stimuli (constant current, cathodic first) were delivered at 0.5 Hz with a wide range of pulse widths (PW in millisecond, 0.02, 0.04, 0.08, 0.1, 0.2, 0.4, 0.6, 0.8, 1, and 2). The evoked action potentials were recorded either by single-fiber recordings from attached L6 DRs10 or by optical GCaMP6f recordings from the DRG somata. To determine the stimulus threshold, the current amplitude was fine adjusted at 0.1 mA resolution so that a pulse train containing 10 stimulus pulses evoked 3 to 5 action potentials.

iv. Determination of Chronaxie

The conduction velocity (CV) determined from single-fiber recordings serves as a criterion to classify DRG neurons into AS and C types. AS-type DRG neurons have CVs between 1 and 7.5 m/s and C type less than 1 m/s. Single-fiber recordings from split L6 nerve roots were filtered (bandpass filter, 300-3000 Hz), digitized at 24 kHz, and stored using the recording module of the TDT system (PZ5-32, RZSD; Tucker-Davis Technologies [TDT], Alachua, Fla.). Single-fiber data were processed off-line using customized MATLAB programs (MathWorks R2019a). The detection threshold of action potentials was set as 4 times the root mean square value of the 5-ms-long noise recorded right before stimulation. Conduction delay was measured between the onsets of stimulus artifacts and the evoked action potentials. Conduction velocity was computed by dividing the distance between the stimulating and recording electrodes with the conduction delay. For GCaMP6f recordings, DRG neurons were grouped by soma size into small-diameter and medium-diameter groups (Φ<20 μm and 20 μm<Φ<35 μm), which generally correlate with C-type and Aδ-type DRG neurons.

The threshold amplitudes of stimulus currents (I) were plotted against stimulus pulse widths (d) to generate the strength-duration curves for both Aδ-type and C-type DRG neurons, which were fitted by a hyperbolic function in Equation 1:

I=b(1+c/d) Equation (1)

where the constants b and c are the rheobase and chronaxie, respectively. Data were presented as means±SE. Student t tests were performed as appropriate using SigmaPlot v9.0 (Systat Software, San Jose, Calif.). Differences were considered significant when P<0.05.

c. Effect of Dorsal Root Ganglion Stimulation on Mouse L6 Spinal Afferent Neural Transmission

The effect of DRG stimulation on colorectal afferent neural transmission was systematically studied in both naive and TNBS-treated mice using an ex vivo preparation in which colorectum, PN, L6 SN, L6 DRG, and L6 DR were harvested in continuity, i.e., an intact colorectum-PN-DRG-DR preparation. Colorectal afferents were activated by colorectal distension (CRD), and evoked action potentials were recorded from split L6 DR filaments by single-fiber recordings. In another series of experiments, the L6 SN, DRG, and DR, i.e., the SN-DRG-DR pathway was preserved to assess the instantaneous change of afferent neural transmission after each stimulus pulse train delivered to L6 DRG, allowing synchronization among action potential generation at SN, DRG stimulation, and single-fiber recording at DR. In the SN-DRG-DR preparation, afferent neural transmission was evoked from the SN by electrical stimulation and recorded at the split L6 DR filaments. Seven naive and 6 TNBS-treated C57BL/6 mice (8-16 weeks, 25-35 g, and either sex) were used for the colorectum-PN-DRG-DR preparation, and 14 naive C57BL/6 mice (8-16 weeks, 25-35 g, and either sex) were used for the SN-DRG-DR preparation. Experimenters were not blinded to intracolonic treatment, but this is not believed to have a significant impact on the objective single-fiber and GCaMP6f recordings from afferents.

i. Ex Vivo Preparation of Colorectum-Pelvic Nerve-Dorsal Root Ganglion-Dorsal Root and Spinal Nerve-Dorsal Root Ganglion-Dorsal Root

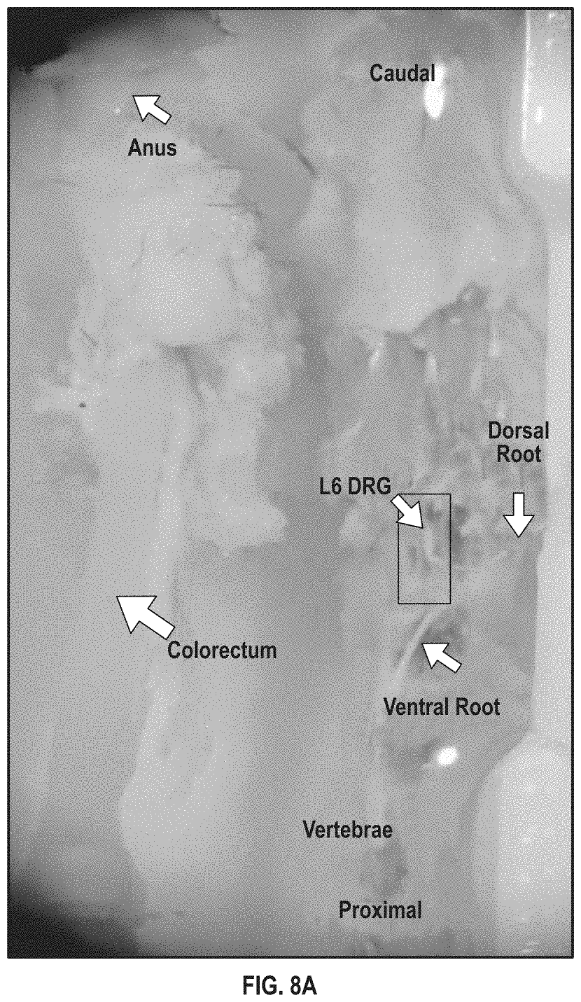

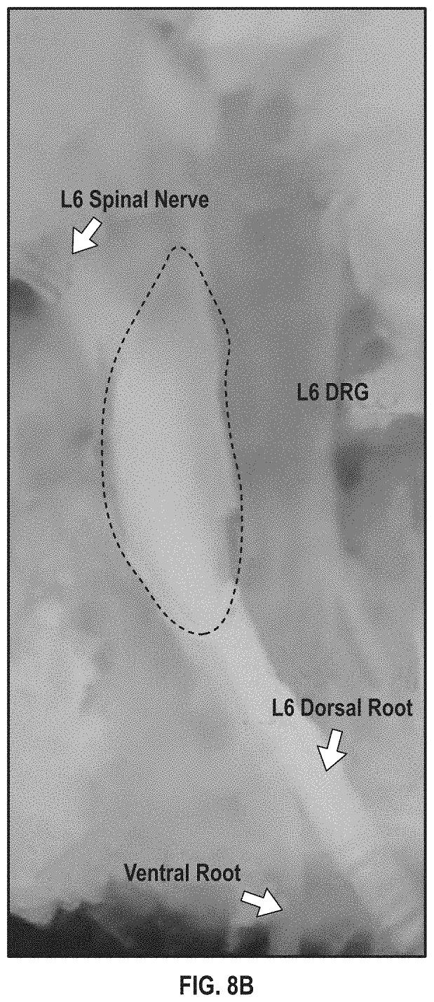

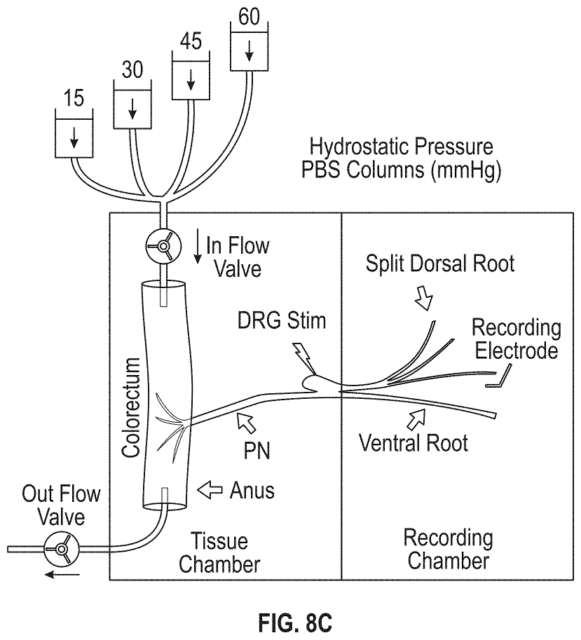

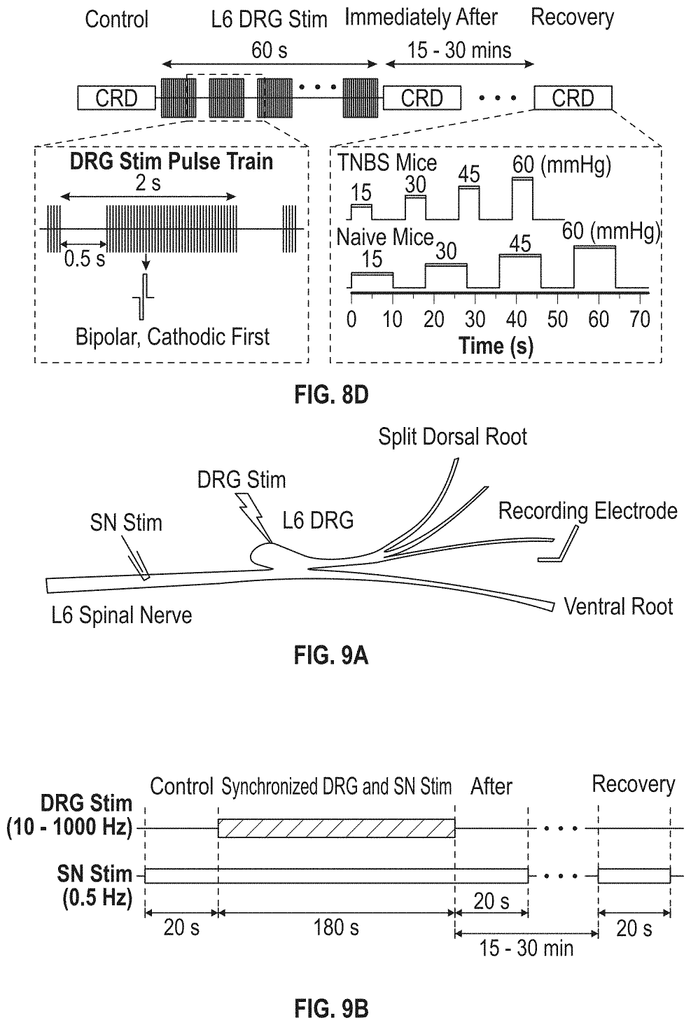

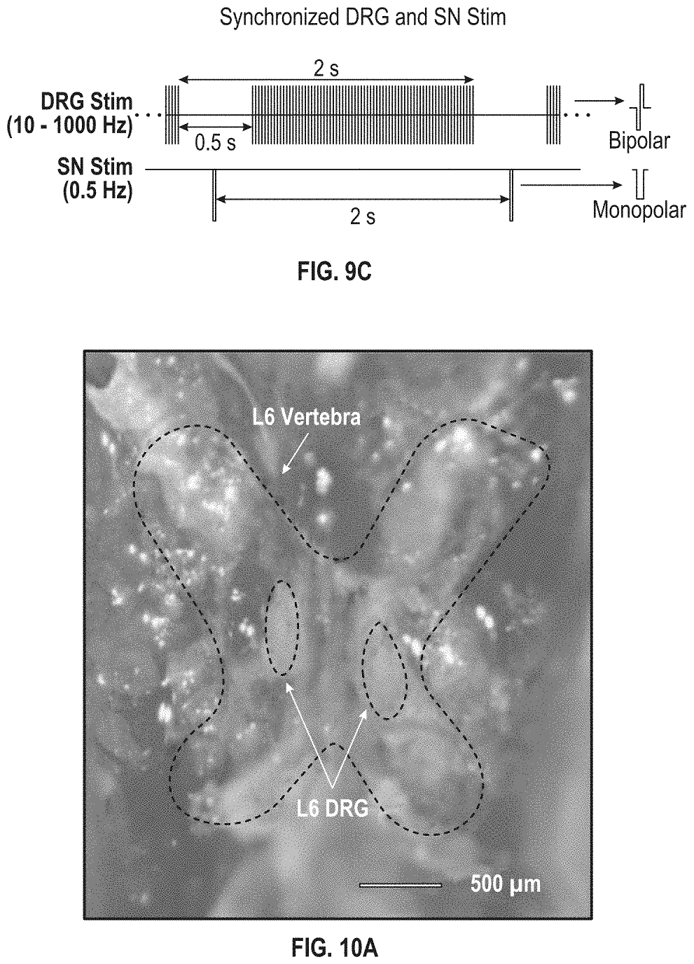

The same procedures described above in the single-fiber recording section were used to perform mouse anesthesia, euthanasia, and dorsal pediculectomy. As shown in FIGS. 8A and 8B, the colorectum-PN-DRG-DR were carefully dissected and placed in a custom-built tissue chamber, which was circulated with oxygenated Krebs solution at 30° C. The L6 DR was gently pulled into the adjacent recording chamber filled with mineral oil and split into fine filaments (˜10 μm) for single-fiber recordings from individual afferent axons using a custom-built microwire electrode array reported previously. As illustrated in FIG. 8C, the colorectum was cannulated and connected to a custom-built CRD device consisting of 4 hydrostatic columns of phosphate-buffered saline (PBS) set at 15, 30, 45 and 60 mm Hg pressures, respectively. Computer-controlled solenoid valves were implemented to regulate the onset and termination of CRD, which reliably evoked neural response in colorectal afferents. In another series of experiments to monitor the effect of DRG stimulation, action potentials were evoked by electrical stimulation of the SN once every 2 seconds and synchronized trains of DRG stimulation were delivered between SN stimulations. Because electrical stimulation bypassed the nerve endings in the colorectum, only the SN-DRG-DR were harvested, as shown in FIG. 9A.

ii. Protocol for Dorsal Root Ganglion Stimulation and Colorectal Distension Using the Colorectum-Pelvic Nerve-Dorsal Root Ganglion-Dorsal Root Preparation

The same needle electrode for characterizing the strength-duration curves described previously was used to deliver constant current stimulation to the caudal region of the L6 DRG, where the somata of high-threshold colorectal afferents are clustered. Biphasic constant current stimuli (charge-balanced bipolar) generated by an IZ2H stimulator (Tucker-Davis Technologies Inc, Alachua, Fla.) were delivered to the L6 DRG at a wide frequency range from 10 to 1000 Hz. Stimulus pulse width was set to be either 0.1 or 0.2 ms based on the chronaxie measurement shown in FIG. 7C. The amplitude of DRG stimulation was set to be suprathreshold so as to evoke action potentials in single-fiber recordings. In some experiments, subthreshold DRG stimulation was also tested during which no action potentials were evoked. As illustrated in FIG. 8D, the CRD protocol was performed before (control), immediately after, and 15 to 30 minutes after DRG stimulation (recovery). A typical DRG stimulation protocol consisted of 30 pulse trains at 0.5 Hz train frequency and 0.5 second intertrain intervals (60 seconds in total). Pulse frequencies for DRG stimulation were set to be one of the following: 10, 50, 100, 500, or 1000 Hz. The CRD protocol consisted of 4 ascending pressure steps of 5 and 10 seconds duration for TNBS-treated and naive mice, respectively, and 8 seconds interstep intervals (15, 30, 45, and 60 mmHg).

iii. Protocol for Synchronized SN and Dorsal Root Ganglion Stimulation with the Spinal Nerve-Dorsal Root Ganglion-Dorsal Root Preparation

The SN-DRG-DR were harvested in continuity as illustrated in FIG. 9A. Neural transmission from the SN to the DR was evoked by electrical stimulation of the SN through a suction electrode pulled from a quartz glass capillary (tip Φ˜300 μm), which delivered cathodic constant current stimulation (0.2 ms pulse width and 0.2-2 mA pulse amplitude) through a stimulus isolator (A365; World Precision Instruments, New Haven, Conn.). To determine the threshold amplitude of DRG stimulation, action potentials in the DR were first evoked by stimulation of the SN or DRG at 0.5 Hz, a low frequency that does not cause marked activity-dependent slowing in unmyelinated C fibers. Stimulating the SN and DRG at 0.5 Hz can evoke activity in the same afferent, as evidenced by identical waveforms in the single-fiber recording but different conduction delays as shown in FIG. 9A. Then, the threshold amplitude of DRG stimulation was determined by stimulating 3 locations along the axial length of the DRG to determine the minimal current amplitude to evoke 3 to 5 action potentials with every 10 stimulus pluses (0.5 Hz). The DRG stimulation location for that afferent was set throughout an experiment. The DRG stimulus intensities were set to be either subthreshold or suprathreshold, corresponding to 70% to 80% and 120% to 150% of the threshold current amplitude, respectively.

To assess the effect of DRG stimulation on afferent neural transmission, the temporally synchronized SN and DRG stimulation protocol was designed as illustrated in FIGS. 9B and 9C. The train frequencies of DRG stimulation and pulse frequency of SN stimulation were both set to be 0.5 Hz. The intertrain interval of DRG stimulation was set to be 0.5 second and in the middle of which SN stimulation was delivered. The synchronized SN and DRG stimulation protocol consisted of 180 seconds of combined SN and DRG stimulation, which was flanked by two 20-second periods of stimulation of the SN alone before (control) and immediately after DRG stimulation. An additional 20 seconds of SN stimulation was conducted 15 to 30 minutes after terminating DRG stimulation (recovery). In each DRG stimulation protocol, pulse frequency was set to be one of the following: 10, 50, 100, 500, or 1000 Hz.

iv. Data Processing

Action potentials evoked by either CRD or SN stimulation were recorded from split DR filaments and processed off-line to identify individual action potentials and compute CV using customized MATLAB programs. The number of action potentials evoked by each CRD protocol was normalized to the number of action potentials evoked by 60 mmHg distension (=100%) in the control trial. Data were presented as means±SE. One-way and Two-way ANOVA and nonparametric Mann-Whitney rank sum or Kruskal-Wallis comparisons were performed, as appropriate, using SigmaPlot v9.0. Differences were considered significant when P<0.05.

d. Effect of Dorsal Root Ganglion Stimulation in Suppressing In Vivo Visceromotor Responses to Colorectal Distension

I. Surgical Preparation for Visceromotor Response Recording and L6 Dorsal Root Ganglion Stimulation

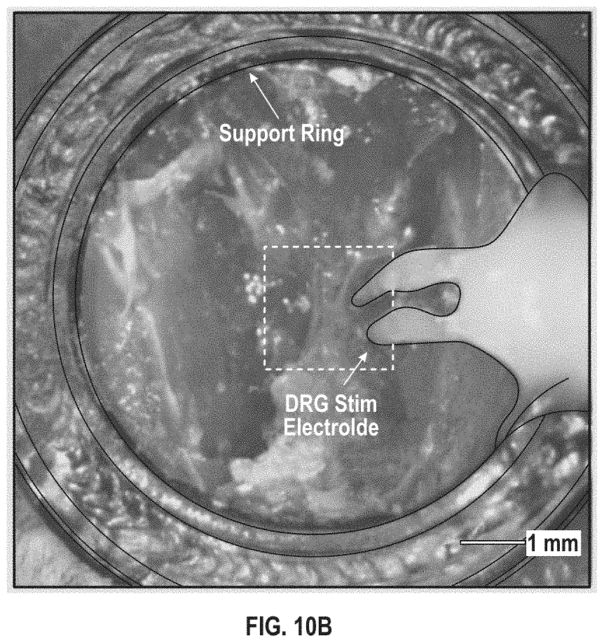

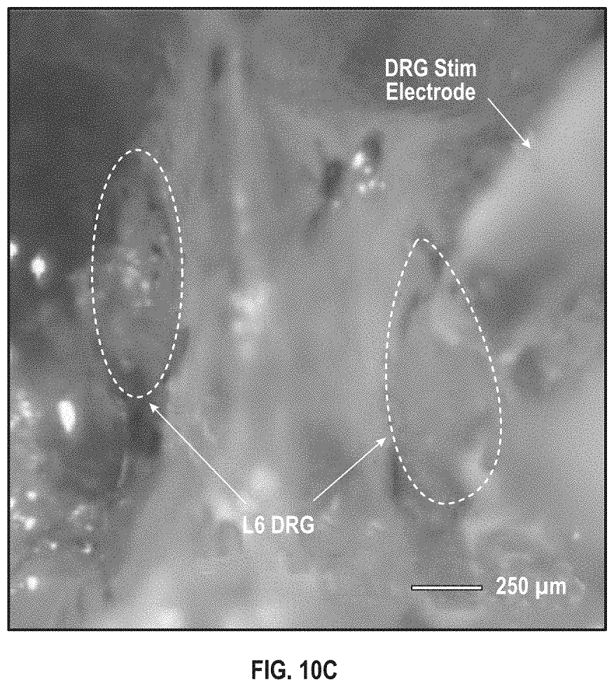

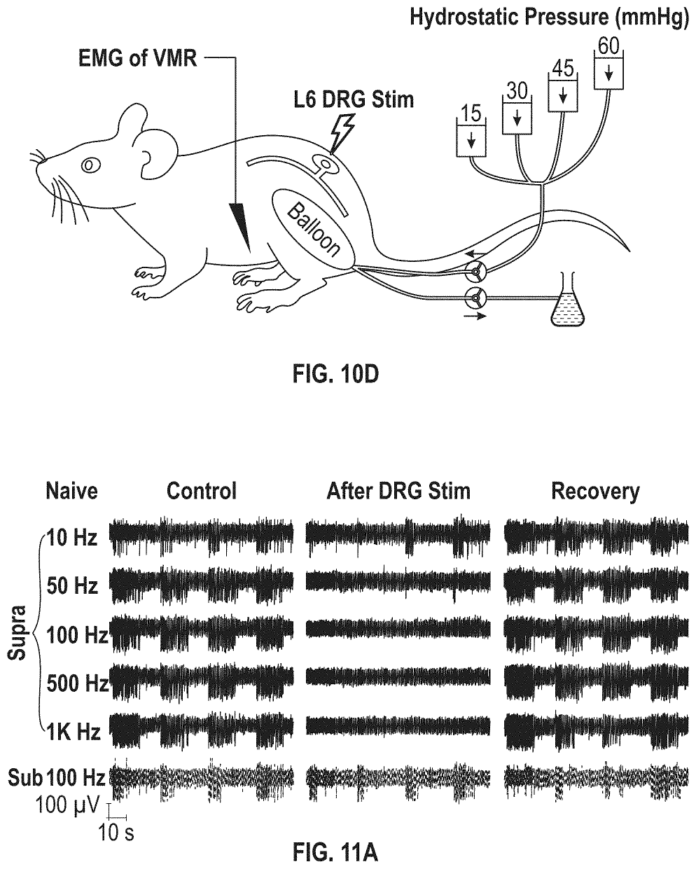

In vivo experiments were conducted on C57BL/6 mice (8-12 weeks of age, 25-35 g, and either sex), which were euthanized right afterwards. First, mouse received one intraperitoneal injection of urethane (1.2 mg/kg), which preserved the spinal-bulbospinal reflex necessary for the behavioral VMRs in rodents. Then, surgical procedures were performed with mouse receiving additional 0.5% to 1% isoflurane anesthesia. A small incision (˜10 mm) was made at the abdominal skin of a mouse in supine position to reveal both left and right oblique musculatures. For each of the 2 oblique muscles, 2 electromyography (EMG) wire electrodes (Teflon-coated stainless steel wire; Cooner Wire, Chats-worth, Calif.) were sutured on (Vicryl suture; Ethicon) and separated by ˜1 mm for measuring the EMG responses to CRD. The abdominal incision was then closed by sutures, and the mouse was flipped to a prone position. The dorsal side of the L6 vertebral segment was carefully exposed by dissecting away the muscle layers as displayed in FIG. 10A. L6 pedicles and the lateral sides of laminae were then removed by trimming the bony structure using a fine-tipped (Φ˜20 μm) dental bur to expose both left and right L6 DRG leaving the spinal canal and articular process intact as shown in FIG. 10A. Special care was taken to avoid perforating the DRG capsule. The isoflurane anesthesia was reduced to 0-0.5% after completing the surgical procedures.

ii. Setup and Protocol for Dorsal Root Ganglion Stimulation and Colorectal Distension In Vivo

A custom-built DRG stimulation electrode was made with platinum-iridium wire (Φ250 Pt80/Ir20; Goodfellow, United Kingdom) and placed in close proximity to L6 DRG for delivering electrical stimuli. To mitigate the movement artefact from respiration and motor reflex, the DRG stimulating electrode was fixed to a ring-shaped adaptor that was glued to the surrounding tissues as shown in FIG. 10B. A magnified view of the exposed L6 DRG and the stimulating electrode is shown in FIG. 10C. The L6 DRGs were covered with Krebs solution to keep the exposed DRG moist and provide electrical conduction for the stimulating electrode. A stimulus isolator (Model 4100; A-M Systems, Sequim, Wash.) was used to deliver biphasic constant current stimuli (charge-balanced bipolar) to the L6 DRG at either 10, 50, or 100 Hz. Stimulus pulse width was set to be either 0.1 or 0.2 ms based on the chronaxie measurement shown in FIG. 7C. The amplitude of DRG stimulation was set to be 120% of the motor threshold, i.e., stimulus threshold at which muscle twitch in limbs or lumbar segment was observed. In some experiments, subthreshold DRG stimulation (60% of the motor threshold) was also tested.

A lubricated polyethylene balloon (1 cm long and 0.6 cm diameter) was inserted into the colorectum and the connecting tube was taped to the tail. The rear end of the balloon was 5 to 10 mm deep inside the colorectum from the anal verge. The balloon was connected to the same custom-built distension device for single-fiber recordings as described above. Electro-myography responses were recorded by a differential amplifier (Model 1700; A-M Systems, Sequim, Wash.) and a digitizer (CED 1401; Cambridge Electronic Design Limited, Cambridge, England). The schematic setup for recording the EMG responses to CRD and conducting L6 DRG stimulation is shown in FIG. 10D. The protocol of conducting CRD and DRG stimulation was similar to in the aforementioned ex vivo single-fiber experiment. Baseline EMG activity was recorded for 10 seconds before CRD, which served as a reference for EMG activity during CRD. The CRD protocol consisted of 4 ascending pressure steps (15,30,45, and 60 mmHg, all at 5 s duration) separated by 8 seconds between steps. To assess the effect of L6 DRG stimulation, VMRs to CRD were recorded before, immediately after, and 15 to 30 minutes after DRG stimulation.

iii. Data Processing

Electromyography activities evoked by CRD were recorded from the abdominal oblique musculature, digitized at 2000 Hz, and processed off-line using customized MATLAB programs. The EMG signals were rectified for calculating the area under the curve (AUC), which was used to evaluate the level of VMR to CRD. Visceromotor responses evoked by CRD were quantified as the AUC values during the 5-second CRD subtracted by a 5-second baseline AUC before the distension. The AUC values from 4 distending pressures (15-60 mmHg) were normalized by the AUC value from 60 mmHg CRD (100%) in the control trial (i.e., without DRG stimulation). Data were presented as means±SE. One-way and Two-way ANOVA and nonparametric Kruskal-Wallis comparisons were performed as appropriate using SigmaStat v4.0. Differences were considered significant when P<0.05.

2. Results

a. Determination of Chronaxie

As summarized in FIG. 7C, stimulus strength-duration curves were measured in 36 DRG neurons from 10 naive mice by single-fiber recordings from split DR filaments, of which 26 were AS type with CV between 1 and 7.5 m/s and 10 were C type with CV less than 1 m/s. The chronaxies were determined to be 105.7±4.0 μs for Aδ-type DRG neurons and 148.9±11.1 μs for C-type DRG neurons. Using GCaMP6f optical recording, 7 naive and 8 TNBS-treated GCaMP6f transgenic mice were used to determine the chronaxie. For the naive GCaMP6f mice, the strength-duration curves of 10 medium-diameter DRG neurons (20 μm<Φ<35 μm) and 12 small-diameter DRG neurons (Φ<20 μm) were quantified; the chronaxies of which were 75.0±5.5 μs and 153.4±11.7 μs, respectively. For the TNBS-treated mice, 20 medium-diameter DRG neurons showed an average chronaxie of 125.0±9.9 μs and 16 small-diameter DRG neurons yielded an average chronaxie of 215.4±13.7 μs.

In naive C57BL/6 or GCaMP6f transgenic mice, the chronaxies of C-type DRG neurons (by single-fiber recordings) and small-diameter DRG neurons (by optical recordings) were not statistically different (t test, P=0.78). The chronaxie values measured from AS-type fibers and medium-diameter DRG neurons differ slightly, but significantly (t test, P<0.001). The chronaxies were significantly different between AS-type and C-type DRG neurons as well as between medium-diameter and small-diameter neurons (t test, P<0.001). 2,4,6-trinitrobenzenesulfonic acid treatment significantly increased the chronaxies for both medium-diameter (t test, P<0.05) and small-diameter neurons (t test, P<0.05) as compared to in naive GCaMP6f mice. To accommodate the stimulation of Aδ-type and C-type DRG neurons, the stimulus pulse width was set at 0.1 or 0.2 ms throughout the experimental study described herein.

b. Suprathreshold Dorsal Root Ganglion Stimulation Effectively Blocks Distension-Evoked (i.e., Mechanical) Afferent Neural Transmission from the Colorectum to the Spinal Cord

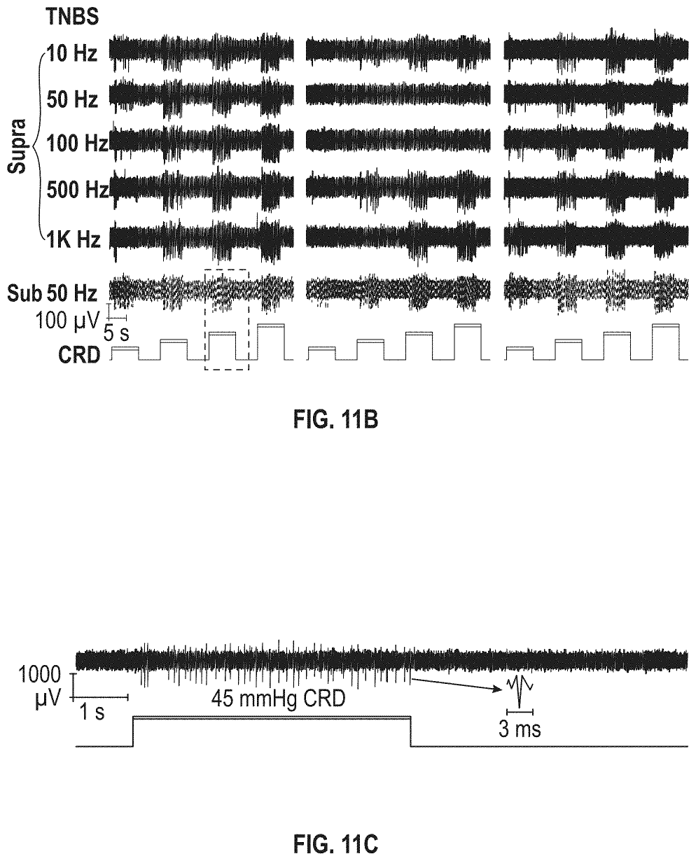

Using the colorectum-PN-DRG-DR preparation, afferent action potentials were evoked by graded CRD and recorded as single units from split DR filaments in both naive and TNBS-treated mice as shown in FIGS. 11A and 11B, respectively. A detailed view of CRD-evoked colorectal afferent activities is displayed in FIG. 11C for the dashed box region in FIG. 11B. As shown in the plots of FIGS. 11A and 11B, studies using a colorectum-PN-DRG-DR preparation that show inhibition of colorectal afferent transmission by suprathreshold DRG stimulation in both naïve (FIG. 11A) and TNBS-treated mice (FIG. 11B). More specifically, FIG. 11A shows transmission of evoked action potentials by CRD is attenuated or completely blocked for naïve mice by suprathreshold DRG stimulation (235 μA, 0.1 ms duration) at all 5 tested frequencies (10, 50, 100, 500 and 1000 HZ) and recovers within 15 to 30 minutes after terminating DRG stimulation. Subthreshold DRG stimulation (150 μA, 0.1 ms duration, 100 Hz) does not block afferent transmission of the same fiber, in contrast to the complete blocking effect by suprathreshold stimulation at 100 Hz. With reference to FIGS. 11B, similar to the results shown in FIG. 11A for naïve mice, suprathreshold DRG stimulation (1 mA, 0.2 ms duration) suppresses transmission of evoked potentials for TNBS-treated mice by CRD at all 5 tested frequencies, which recovers after terminating the DRG stimulation. Subthreshold DRG stimulation (700 μA, 0.2 ms duration, 50 Hz) does not block transmission in the same afferent fiber. Thus, the neural transmission in colorectal afferents was blocked or attenuated immediately after suprathreshold DRG stimulation at all 5 tested pulse frequencies (10,50,100,500, and 1000 Hz). By contrast, subthreshold DRG stimulation of 50 and 100 Hz did not block colorectal afferent transmission in either naive or TNBS-treated mice, frequencies at which complete conduction block was achieved at suprathreshold stimulation.

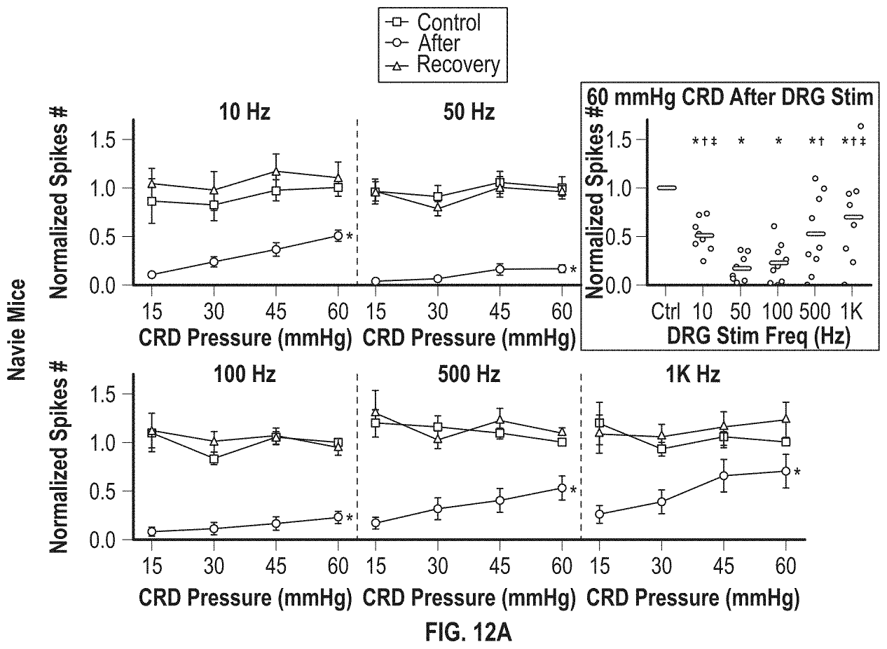

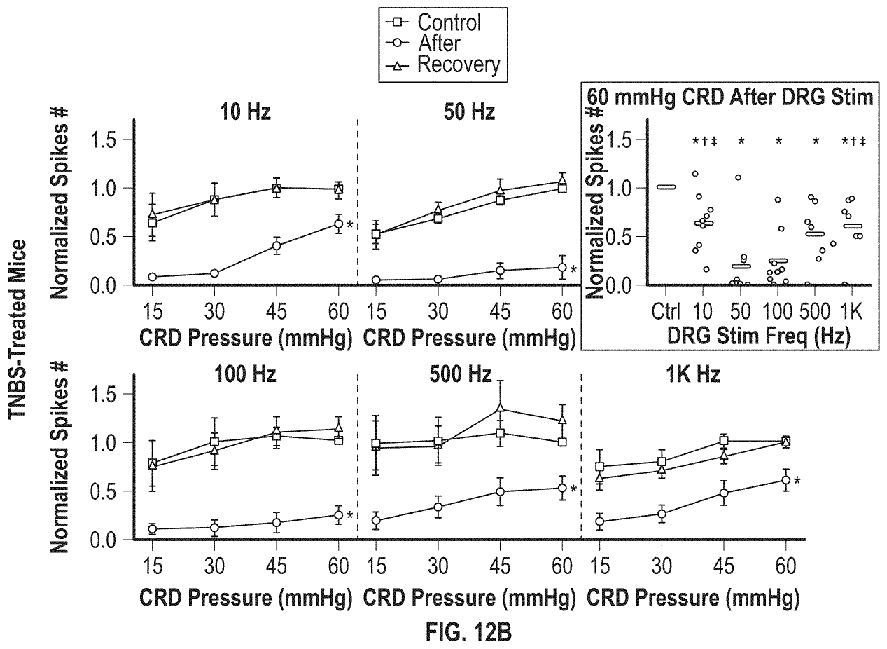

Suprathreshold DRG stimulation was assessed in 9 colorectal afferents from 7 naive mice and 9 afferents from 6 TNBS-treated mice as shown in FIGS. 12A and 12B, where the number of evoked spikes was normalized to the number of spikes evoked by the 60 mm Hg pressure step in control trials before DRG stimulation. Immediately after DRG stimulation, responses to CRD were significantly reduced at all 5 tested frequencies of DRG stimulation (10, 50, 100, 500 and 1000 Hz) for both naive and TNBS-treated mice as shown in FIGS. 12A and 12B, respectively (Two-way ANOVA, Bonferroni post hoc comparison, P<0.001 for control vs after in naive and TNBS-treated mice). Responses to CRD recovered completely 15 to 30 minutes after terminating DRG stimulation (post hoc comparison for control vs recovery, P>0.5 in naive mice and P>0.25 in TNBS-treated mice for all 5 frequencies). Colorectal distension responses at 60 mmHg pressure were normalized and were plotted in insets for FIGS. 12A and 12B after DRG stimulation at all 5 frequencies (10, 50, 100, 500, and 1000 Hz) for naive and TNBS-treated mice, respectively. DRG stimulation by all 5 frequencies significantly attenuated afferent responses as compared to control (one-way ANOVA, P<0.05 for control vs 10, 50, 100, 500, and 1000 Hz in both naive and TNBS-treated mice). The inhibitory effect of 50 Hz DRG stimulation was significantly greater than that of 10, 500, or 1000 Hz in naive mice (one-way ANOVA, P<0.025 for 50 vs 10, 500, and 1000 Hz). In TNBS-treated mice, the inhibitory effect of 50 Hz stimulation was significantly greater than that of 10 or 1000 Hz (one-way ANOVA, P<0.047 for 50 vs 10 and 1000 Hz). Similarly, 100 Hz stimulation showed a significantly greater inhibitory effect than 10 or 1000 Hz in both naive and TNBS-treated mice (one-way ANOVA, P<0.05 for 100 vs 10 or 1000 Hz). There was no statistical difference between 50 and 100 Hz or between 100 and 500 Hz stimulations in either naive or TNBS groups (P=0.527 for 50 vs 100 Hz, P=0.063 for 100 vs 500 Hz in naive mice; P=0.305 for 50 vs 100 Hz, P=0.096 for 100 vs 500 Hz in TNBS-treated mice). Comparing the naive and TNBS groups, there was no significant difference in the inhibitory effect of DRG stimulation to suppress the transmission of spikes evoked by 60 mm Hg distension (one-way ANOVA, P>0.17). In addition, the recovered responses to 60 mmHg CRD at 15 to 30 minutes after DRG stimulation were not significantly different between naive and TNBS groups (one-way ANOVA, P>0.29).

c. Suprathreshold Dorsal Root Ganglion Stimulation Progressively Increased Afferent Conduction Delay and Led to Conduction Block

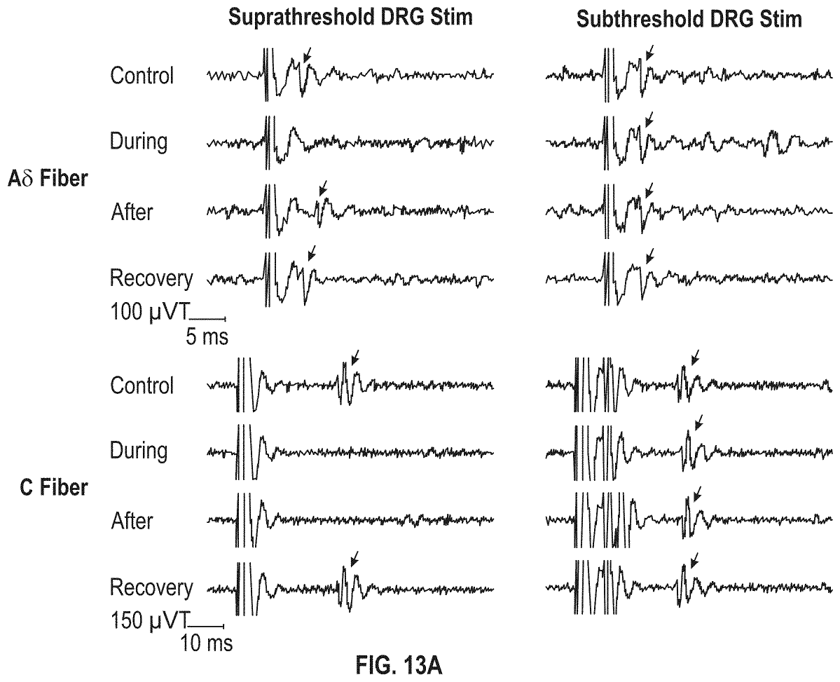

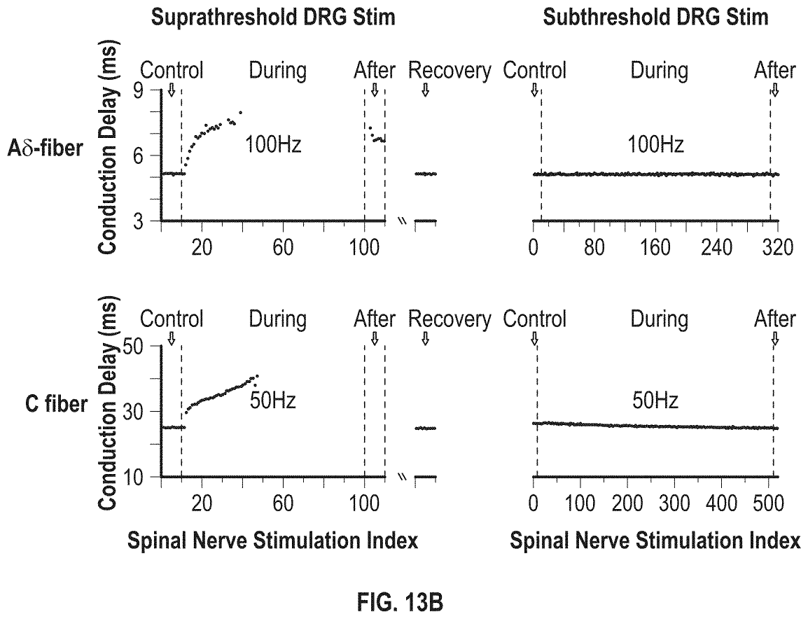

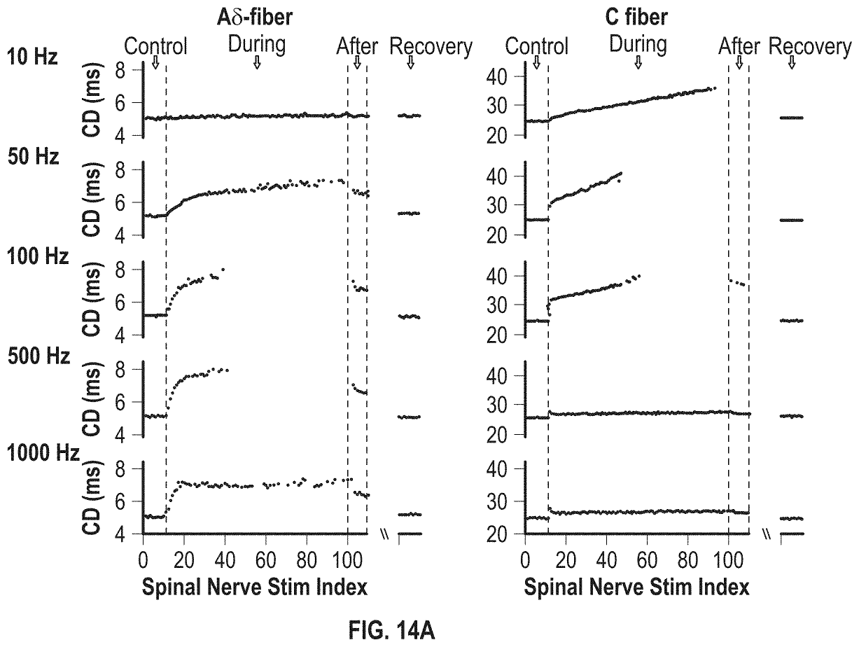

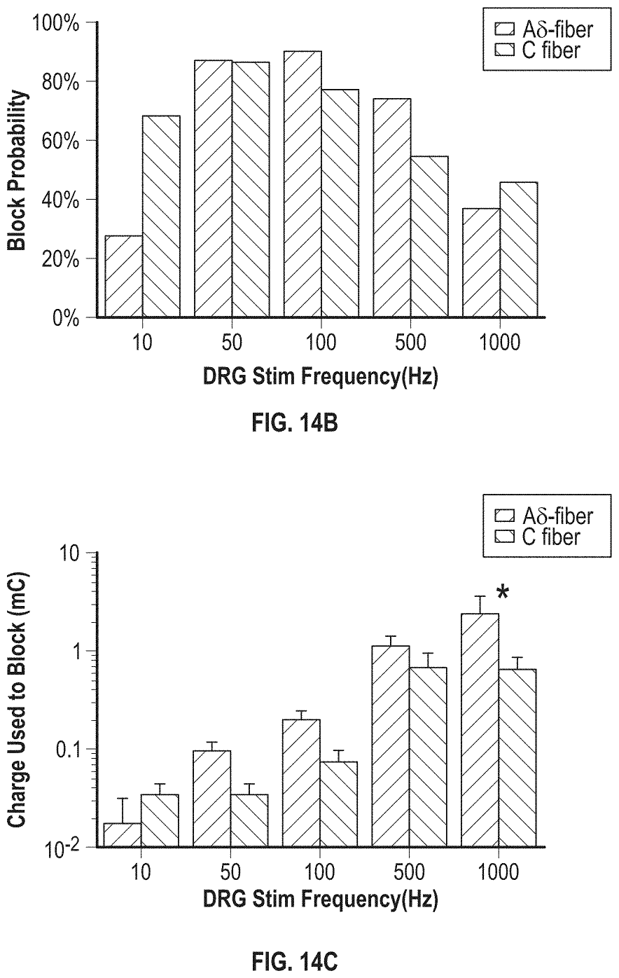

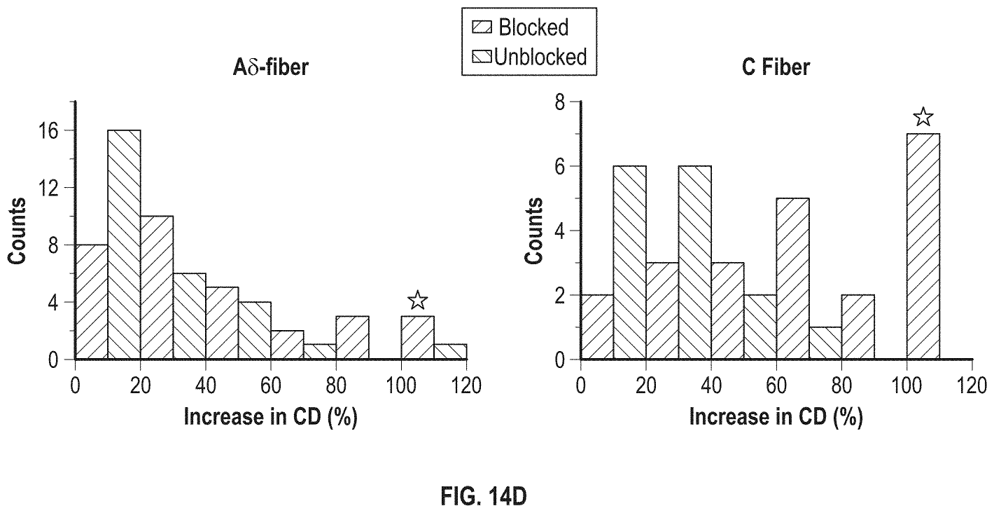

The neuromodulatory effect of DRG stimulation as assessed immediately after the DRG stimulation is shown in FIGS. 13A, 13B and 14A-14D. The mechanical CRD stimuli according to the present disclosure presented significant technical challenges to study the instantaneous effect of DRG stimulation on afferent transmission during the course of CRD. The challenge was addressed by using a SN-DRG-DR preparation as described in the Methods section, which allowed instantaneous monitoring of afferent transmission during DRG stimulation. As shown by single-fiber recordings from typical Aδ and C fibers in FIG. 13A, action potentials were evoked by SN stimulation and recorded at L6 DRs. The conduction delay (CD) of individual afferents was assessed before (as control), during, and after DRG stimulation once every 2 seconds. Spinal nerve stimulation was applied once every 2 seconds; each one of the SN stimulation was assigned with a stimulation index number. The CD of a typical Aδ fiber and a C fiber was plotted against the SN stimulation index and displayed in FIG. 13B. The CD increased instantaneously and progressively during suprathreshold DRG stimulation, and both afferents in FIGS. 13A and 13B eventually failed to conduct action potentials after suprathreshold DRG stimulation. Afferent transmission was fully recovered 15 to 30 minutes after terminating suprathreshold DRG stimulation. The effect of subthreshold DRG stimulation (70%-80% of the threshold current amplitude) on afferent neural transmission was also assessed using the same synchronized SN and DRG stimulation protocol. As shown in FIG. 13B, 50 and 100 Hz subthreshold DRG stimulation, frequencies that demonstrated optimal blocking efficiency by suprathreshold stimulation (see insets in FIGS. 12A and 12B) did not affect conduction delay or block afferent transmission even after 15 minutes of DRG stimulation.

The neuromodulatory effects of suprathreshold DRG stimulation were studied in 31 Aδ-type afferents (N=29 for 10 Hz; N=30 for 1000 Hz; N=31 for 50,100, and 500 Hz) and 22 C-type afferents at 5 stimulus frequencies (10, 50, 100, 500 and 1000 Hz) from 14 mice; the results of which are shown in FIGS. 14A-14D. The blocking effects of suprathreshold DRG stimulation (10-1000 Hz) on atypical AS fiber and a C fiber are displayed in FIG. 14A. A complete afferent transmission block was defined as the continuous absence of action potentials in the recording for at least 20 seconds (10 SN stimulations). As shown in FIG. 14B, complete transmission block is frequency dependent. Dorsal root ganglion stimulation at 50, 100 and 500 Hz blocked 87%, 90%, and 74% of the tested Aδ fibers, respectively. In comparison, 10 and 1000 Hz stimulation only blocked 27% and 36% of Aδ fibers. Similarly, C fibers were preferentially blocked by 50 and 100 Hz DRG stimulation (86% and 77%), rather than by lower (10 Hz at 68%) or higher (500 Hz at 54% and 1000 Hz at 45%) frequencies. Dorsal root ganglion stimulation at 10 Hz blocked a significantly greater proportion of C fibers than Aδ fibers (Fisher exact test, P=0.005).

Frequencies above 10 Hz showed no statistical difference in blocking Aδ and C fibers (Fisher exact test, P=1, 0.253, 0.155, and 0.576 for 50, 100, 500, and 1000 Hz, respectively).

In afferents with complete transmission block, the cumulative positive electrical charge delivered to the DRG before a complete block was calculated by integrating the positive part of the stimulus pulses (charge balanced) from the onset of DRG stimulation to the onset of transmission block. The total charge to block is displayed in FIG. 14C, which shows a monotonous increase with increased stimulus frequency. The required charge to block Aδ fibers was significantly greater than that required to block C fibers only at 1000 Hz stimulation (Two-way ANOVA, Aδ vs C fibers, P=0.005; post hoc comparison, P=0.004 for 1000 Hz and P>0.9 for other frequencies).

Typically, the increase in conduction delay peaked right before the complete afferent block (FIG. 14A for 100 Hz stimulation). The maximum increase in CD for each afferent was measured in both blocked and unblocked afferents and is shown in FIG. 14D. Some afferents were blocked after the first 1.5-second-long pulse train of DRG stimulation, and their CD increase could not be quantified and so were arbitrarily assigned to be the upper boundary (120%) and marked with stars in FIG. 14D. The average maximum CD increase was 39.1±4.4% in blocked Aδ fibers, significantly higher than that in unblocked Aδ fibers (25.6±4.9%, one-way ANOVA, P=0.046). Similarly, the average maximum CD increase was significantly higher in blocked C fibers (51.9±5.9%) than that in unblocked C fibers (27.9±4.4%, P=0.003). Between Aδ and C fibers, there were not statistical differences in maximum CD increase within either blocked or unblocked groups (one-way ANOVA, P=0.094 and 0.765 for blocked and unblocked groups, respectively).

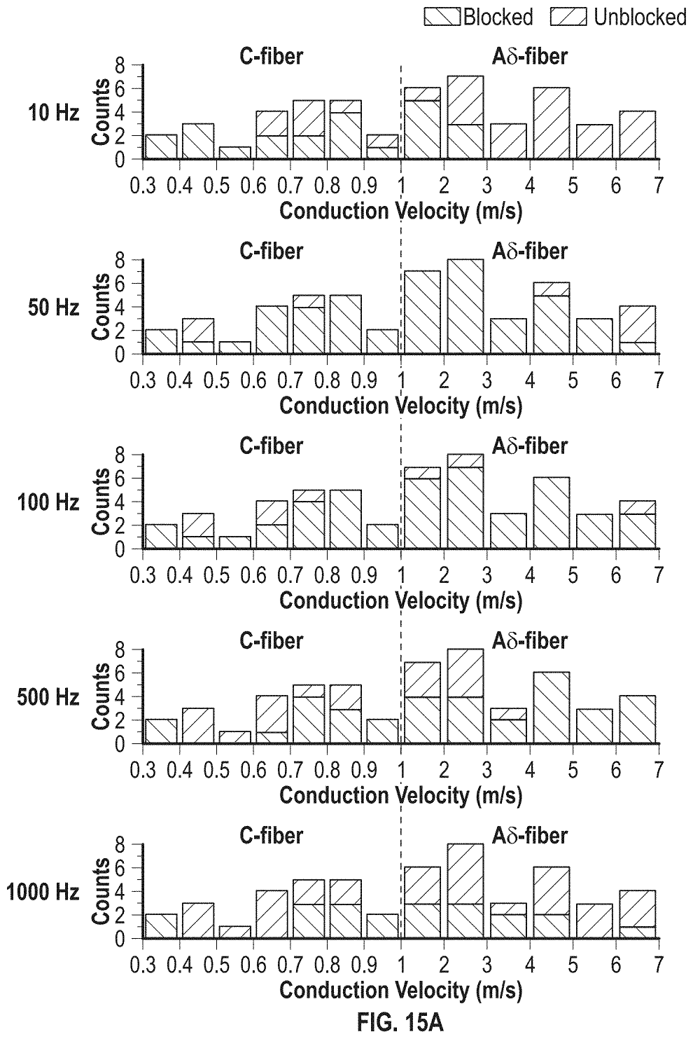

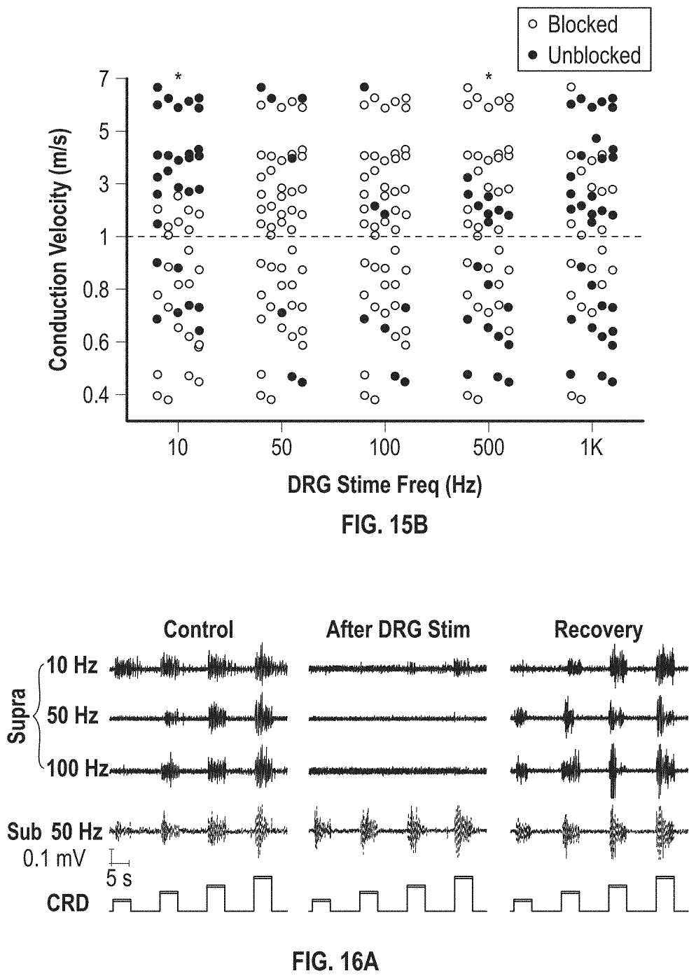

The histogram of baseline CV from 31 Aδ fibers and 22 C fibers subjected to DRG stimulation is displayed in FIG. 15A, and the scatter plot of those afferents at different CV undergoing DRG stimulation at all 5 tested frequencies is shown in FIG. 15B. The data shows an apparent dependency in blocking effect of DRG stimulation based on both the stimulus frequency and the CV of the individual afferent. Stimulus frequencies of 50 and 100 Hz efficiently blocked greater than 85% of tested afferents across the entire range of CVs (0.3-7 m/s). There is no statistical difference in CV between the blocked and unblocked afferents at 50 and 100 Hz (Mann-Whitney rank-sum test, P 5 0.503 and 0.115 at 50 and 100 Hz, respectively). Lower and higher stimulus frequencies were not efficient in blocking afferents at certain CV as shown in FIGS. 15A and 15B. At 10 Hz DRG stimulation, only slower conducting afferents were blocked and none of the afferents with CV faster than 3 m/s were blocked. The average CV of blocked afferents by 10 Hz stimulation is significantly slower than the unblocked counterpart (Mann-Whitney rank-sum test, P<0.001). Similarly, 500 Hz stimulation preferentially blocked afferents with CV greater than 0.7 m/s, and the average CV of blocked afferents is faster than that of unblocked counterpart (Mann-Whitney rank-sum test, P=0.007). For 1000 Hz DRG stimulation, there is no statistical difference in average CV between blocked and unblocked afferents (Mann-Whitney rank-sum test, P=0.654).

d. Suprathreshold L6 Dorsal Root Ganglion Stimulation (10-100 Hz) Significantly Attenuates In Vivo Visceromotor Responses to Colorectal Distension

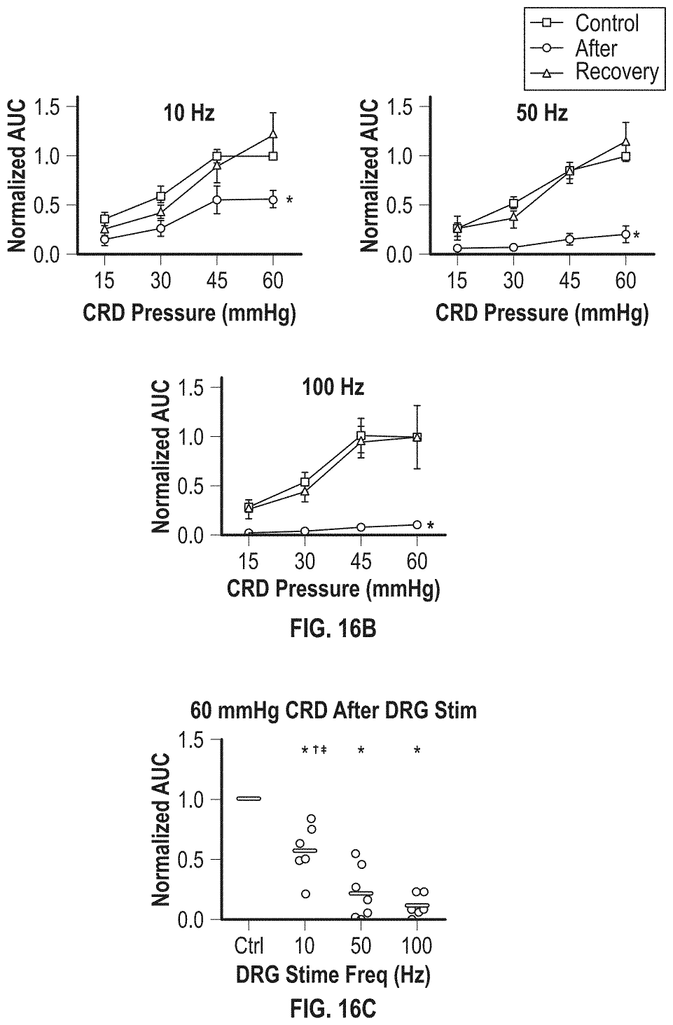

In urethane-anesthetized mice, the VMR to CRD as a metric of noxious visceral stimulus was quantified by EMG activities recorded from the abdominal oblique musculature as shown in FIG. 16A. The EMG activities during CRD were attenuated immediately after the suprathreshold DRG stimulation at 10, 50, and 100 Hz. The EMG responses recovered 15 to 30 minutes after terminating the DRG stimulation. By contrast, subthreshold DRG stimulation had no apparent effect on VMR to CRD as shown in the gray traces in FIG. 16A. Displayed in FIG. 16B is the summarized results of in vivo DRG stimulation from 7 mice. The AUC of rectified EMG signals was subtracted by the baselined AUC before the distension and normalized to the AUC of EMG signal from 60 mm Hg distension in control trials before DRG stimulation. The AUC values of EMG responses evoked by CRD were significantly reduced immediately after DRG stimulation at all 3 tested frequencies (Two-way ANOVA, Bonferroni post hoc comparison, P<0.001 for control vs after at 10, 50, and 100 Hz). The AUC of EMG responses recovered completely 15 to 30 minutes after DRG stimulation (post hoc comparison for control vs recovery, P=1 for all 3 frequencies). The AUC values of EMG response to 60 mmHg pressure CRD were normalized and plotted in FIG. 16C after 10, 50, and 100 Hz DRG stimulation, which revealed significant inhibition of VMR to CRD by DRG stimulation (one-way ANOVA, P<0.001 for control vs 10, 50, and 100 Hz, respectively). The inhibitory effects of 50 and 100 Hz DRG stimulation were significantly greater than 10 Hz (one-way ANOVA, P<0.001 for 100 vs 10 Hz, P=0.004 for 50 vs 10 Hz). The inhibitory effect between 50 and 100 Hz was not significantly different (one-way ANOVA, P=0.628).

e. Discussion

Based on the experimental results presented herein, it is clear that reversible peripheral nerve block by electrical stimulation offers significant clinical benefits. Anodal nerve block with direct current stimulation leads to imbalanced electrochemical reactions at the electrode-tissue interface and is best reserved as a research tool to selectively block myelinated axons. Charge-balanced kilohertz stimulation (generally 1-30 kHz) reversibly blocks peripheral nerves with rapid onset, modest carry-over effects and no apparent tissue damage, which has demonstrated efficacy in clinical applications. By contrast, the relatively few reports on nerve block using subkilohertz stimulation are found either to implement charge-unbalanced stimulation (see, Solomonow M, Eldred E, Lyman J, Foster J. Fatigue considerations of muscle contractile force during high-frequency stimulation. Am J Phys Med 1983; 62:117-22) or to cause no conduction block but depletion of neural transmitters (see, Dowden B R, Wark H A, Normann R A. Muscle-selective block using intrafascicular high-frequency alternating current. Muscle Nerve 2010; 42: 339-47).

By using in vivo single-fiber recordings, a recent report did show that 20 Hz DRG stimulation effectively blocks neural transmission in rodent somatic afferents (see, Chao D, Zhang Z, Mecca C M, Hogan Q H, Pan B. Analgesic dorsal root ganglionic field stimulation blocks conduction of afferent impulse trains selectively in nociceptive sensory afferents. PAIN 2020; 161:2872-86). Consistent with Chao et al. study, the experimental studied presented herein show that visceral afferents can also be blocked by subkilohertz charge-balanced stimulation at the DRG. The experimental results presented herein also confirm attenuation of reflex response to noxious visceral stimulation, and thus support DRG stimulation as a beneficial therapeutic approach to visceral pain.

As demonstrated herein, DRG stimulation at 50 Hz efficiently blocks neural transmission in greater than 85% of thinly myelinated AS-type and unmyelinated C-type afferents, which can be potentially applied for managing pain, e.g., from pelvic visceral organs pre-dominantly innervated by C fibers and Aδ fibers, including the urinary bladder, uterus, prostate, colon, and rectum. The DRG stimulus amplitude may be advantageously set slightly above the threshold, e.g., 20%-50% above the threshold, to provide an intensity unlikely to cause permanent neural damage. In support, the conduction of both electrically and mechanically evoked action potentials recovers completely within 15 to 30 minutes after terminating DRG stimulation.

Of note, previous reports generally quantify the effect of neuro-modulation by either compound action potentials (CAPs) or physiological functions of attached organs (e.g., bladder pressure or muscle forces). [See, e.g., Patel Y A, Butera R J. Challenges associated with nerve conduction block using kilohertz electrical stimulation. J Neural Eng 2018; 15:031002; Zhang Z, Lyon T D, Kadow B T, Shen B, Wang J, Lee A, Kang A, Roppolo J R, de Groat W C, Tai C. Conduction block of mammalian myelinated nerve by local cooling to 15-30″C after a brief heating. J Neurophysiol 2016; 115:1436-45] Compound action potentials as temporal and spatial summations of neural activities from bulk nerve bundles are not sensitive enough to detect neuromodulatory effects on individual axons. In particular, slow-conducting C and Aδ fibers are underrepresented in the amplitudes of CAPs compared with Act and AP fibers, rendering CAPs unsuitable for studying slow-conducting visceral afferents. Physiological functions allow convenient assessment of efferent nerve block, but cannot be applied to studying sensory afferents.

The present disclosure provides strength-duration curves from intact DRG through single-fiber recordings and GCaMP6f recordings to establish an appropriate stimulus pulse width near the chronaxie to achieve better energy efficiency of neural stimulation. Single-fiber recordings from attached DRs allow classification of afferents into C and Aδ types based on CV. Using GCaMP6f recordings, strength-duration curves may be measured in small-diameter (<20 μm) and medium-diameter (20-35 μm) DRG neurons and the may be classified as C-type and Aδ-type afferents because rodent DRG soma size correlates with axon myelination. Chronaxies determined using both methods are comparable and not statistically different. The short chronaxies (75-215 μs) indicate that spike initiation by DRG stimulation is unlikely to be in the somata but to be at attached axons because chronaxies of neural somata are usually longer than 1 ms. Indeed, chronaxies are longer than 2 ms for dissociated human DRG neurons and shorter than 665 μs for human sensory nerves.

It is noteworthy that the above reported chronaxies were measured from dissociated DRG neurons, which may have altered ion channel composition and density from neurons in intact DRG. In the central nervous system, chronaxies are much shorter in axons (30-700 μs) than those in somata (1-10 ms). In addition, we observe in GCaMP6f recordings that it is usually not the neurons next to the stimulus electrode, but those further from the electrode are evoked by threshold DRG stimulation. Collectively, the results indicate that DRG stimulation outside the dura mater likely activates the attached axons for spike initiation, rather than the neural somata.

It is believed that the conduction block produced by DRG stimulation occurs at the t-junction based on 2 categories of evidence. First, spike transmission from the peripheral to the central axons (i.e., through-conduction) does not require bifurcating propagation into the soma, and changes in soma conductance have minimal effects on the through-conduction. Hence, blocking the stem region or soma likely has no appreciable effects on the through-conduction. Second, the safety factor for spike through-conduction is the lowest at the t-junction, as supported by substantial experimental evidence. Computational simulations further confirm that propagation failure likely occurs at bifurcating points, such as the t-junction. Suprathreshold, not sub-threshold, DRG stimulation blocks afferent conduction, which suggests an activity-dependent mechanism of conduction block at the t-junction.

Extensive studies have been conducted to reveal a low-pass “filtering” function of the t-junction that causes conduction failure at higher spiking frequencies. Repetitive spiking can cause depolarization in some DRG somata and hyperpolarization in others. Neither soma hyperpolarization nor depolarization seems to have direct influence on through-conduction at the t-junction. Studies focusing on Ca21 concentrations indicate that the “filtering” function of the t-junction is calcium-dependent. Thus, membrane hyperpolarization likely occurs at the t-junction through Ca2+-mediated ion channels, e.g., Ca2+-activated K+ channels.

In addition, an increase in Na+ concentration is apparent even after a few action potentials in the confined axonal space, which can hyperpolarize the t-junction membrane by reducing the Na+ reversal potential. In addition, persistent soma hyperpolarization can electronically hyperpolarize the t-junction membrane of those afferents with short-diameter and large-diameter stem axons as reported by a theoretical study. [See, Sundt D, Gamper N, Jaffe D B. Spike propagation through the dorsal root ganglia in an unmyelinated sensory neuron: a modeling study. J Neurophysiol 2015; 114:3140-53] In support, pharmacological hyperpolarization of soma membrane potential enhances afferent conduction failure and reduces rodent behavioral responses to noxious paw stimuli. Synchronized DRG and nerve stimulation according to the present disclosure reveals a monotonous activity-dependent slowing of conduction velocities after DRG stimulation, which also lends support to hyperpolarization at the t-junction for conduction block. Notably, Aδ and C fibers blocked by DRG stimulation show significantly greater increases in conduction delay than their unblocked counterparts (see, FIG. 14D). Collectively, evidence presented herein implicates membrane hyperpolarization as a plausible mechanism for activity-dependent conduction block at the t-junction.

In the systems and methods of the present disclosure, mid-range DRG stimulation frequencies (50-500 Hz) are more efficient in blocking conduction than the lower (10 Hz) or higher (1 kHz) frequencies tested. Previous research indicates pronounced activity-dependent slowing in conduction velocities in nociceptive C fibers when stimulus frequency is greater than 2 Hz. As shown in FIGS. 15A and 15B, 10 Hz DRG stimulation selectively blocks C fibers and some slower-conducting AS fibers, whereas none of the AS fibers with CVs greater than 3 m/s are blocked. This is likely due to the prominent activity-dependent slowing in C fibers. By contrast, AS fibers with CVs greater than 3 m/s show limited activity-dependent slowing at 10 Hz DRG stimulation (data not shown), which agrees with previous observations that activity-dependent slowing is not prominent in fast-conducting A fibers. At 100 and 500 Hz stimulation, progressive increases in conduction delay (i.e., slowing) in AS fibers is noted, which likely accounts for the block effects in those fibers. For C fibers, DRG stimulation beyond 50 Hz results in a gradual reduction in blocking probability. This is likely because C fibers fire at less than 20 Hz physiologically and cannot follow higher stimulation frequency.

Similarly, blocking probability is reduced for Aδ fibers with further increases in stimulus frequency beyond 500 Hz. Stimulation in the kilohertz range generally does not reliably evoke action potentials and is considered sub-threshold stimulation. The blocking effect of 50 to 100 Hz

DRG stimulation was further validated by in vivo recordings of VMRs to CRD, a pseudoeffective reflex response widely used in the literature to quantify the level of noxious visceral stimulation in rodents, thereby objectifying the experience of visceral pain. The in vivo outcome that 50 and 100 Hz DRG stimulation has significantly greater inhibition on VMR than 10 Hz stimulation well agrees with the findings from ex vivo single-fiber recordings. Efforts to assess the in vivo effect of 500 and 1000 Hz DRG stimulation were unsuccessful because suprathreshold stimulation at those high frequencies evoked tetanic contraction of limb muscles to confound the VMRs.

In summary, the experimental results presented herein demonstrate that L6 DRG stimulation effectively blocks distension-evoked (i.e., mechanical) afferent neural transmission from the colorectum to the spinal cord in the subkilohertz range (10-1000 Hz); 50 and 100 Hz stimulation produce superior blocking probability as compared to stimulation at 10, 500, or 1000 Hz. Using synchronized DRG and L6 SN stimulation, it is noted that DRG stimulation causes activity-dependent conduction slowing in both C-type and AS-type afferents. Afferents blocked by DRG stimulation exhibit a significantly greater increase in conduction delay than unblocked counterparts. Mid-range stimulation frequencies block conduction more efficiently and produce greater activity-dependent slowing than either low (10 Hz) or high (1000 Hz) frequency stimulation. Indeed, the data herein shows that the average chronaxie of L6 DRG stimulation is below 216 μs for C fibers and 125 μs for AS fibers, which indicates spike initiation likely at the attached afferent axons rather than in somata. The monotonous and progressive increase in conduction delay during DRG stimulation supports hyperpolarization of the t-junction as the underlying mechanism of conduction block. The blocking effect of 50 and 100 Hz DRG stimulation is further validated by an in vivo experiment showing suppressed VMRs to CRD in urethane-anesthetized mice. The results reported here demonstrate a mechanism of afferent modulation that subkilohertz DRG stimulation is capable of blocking conduction in C and AS fibers, a promising neuromodulation strategy for managing chronic visceral pain.

### REFERENCE INFORMATION

As used herein, “a”, “an”, and “the” refer to both singular and plural referents unless the context clearly dictates otherwise.

As used herein, the term “about” refers to a measurable value such as a parameter, an amount, a temporal duration, and the like and is meant to include variations of +/−15% or less, preferably variations of +/−10% or less, more preferably variations of +/−5% or less, even more preferably variations of +/−1% or less, and still more preferably variations of +/−0.1% or less of and from the particularly recited value, in so far as such variations are appropriate to perform in the invention described herein. Furthermore, it is also to be understood that the value to which the modifier “about” refers is itself specifically disclosed herein.

As used herein, spatially relative terms, such as “beneath”, “below”, “lower”, “above”, “upper”, “front”, “back”, “side”, “left”, “right”, “rear”, and the like, are used for ease of description to describe one element or feature's relationship to another element(s) or feature(s). It is further understood that the terms “front”, “back”, “left”, and “right” are not intended to be limiting and are intended to be interchangeable, where appropriate. Further, it should be noted that the terms “first,” “second,” and the like herein do not denote any order, quantity, or relative importance, but rather are used to distinguish one element from another.

As used herein, the terms “comprise(s)”, “comprising”, and the like, specify the presence of stated features, integers, steps, operations, elements, and/or components, but do not preclude the presence or addition of one or more other features, integers, steps, operations, elements, components, and/or groups thereof.

As used herein, the terms “configure(s)”, “configuring”, and the like, refer to the capability of a component and/or assembly, but do not preclude the presence or addition of other capabilities, features, components, elements, operations, and any combinations thereof.

Chemical compounds are described using standard nomenclature. For example, any position not substituted by any indicated group is understood to have its valency filled by a bond as indicated, or a by hydrogen atom.

All ranges disclosed herein are inclusive of the endpoints, and the endpoints are independently combinable with each other. Each range disclosed herein constitutes a disclosure of any point or sub-range lying within the disclosed range.

All methods described herein can be performed in any suitable order unless otherwise indicated herein or otherwise clearly contradicted by context. The use of any and all examples, or exemplary language (e.g., “such as”), is intended merely to better illustrate the invention and does not pose a limitation on the scope of the invention or any embodiments unless otherwise claimed.

Any combination or permutation of features, functions and/or embodiments as disclosed herein is envisioned. Additional advantageous features, functions and applications of the disclosed systems, methods and assemblies of the present disclosure will be apparent from the description which follows, particularly when read in conjunction with the appended figures. All references listed in this disclosure are hereby incorporated by reference in their entireties.

While the invention has been described with reference to preferred embodiments, it will be understood by those skilled in the art that various changes may be made and equivalents may be substituted for the elements thereof without departing from the scope of the invention. In addition, many modifications may be made to adapt the teaching of the invention to particular use, application, manufacturing conditions, use conditions, composition, medium, size, and/or materials without departing from the essential scope and spirit of the invention. Therefore, it is intended that the invention is not limited to the exemplary embodiments and best mode contemplated for carrying out this invention as described herein. Since many modifications, variations, and changes in detail can be made to the described examples, it is intended that all matters in the preceding description and shown in the accompanying figures be interpreted as illustrative and not in a limiting sense.

1. A system for electrical stimulation to effectuate selective sensory transmission block, comprising:
a. one or more stimulating electrodes adapted to deliver electrical simulation to one or more targeted afferents within an intervertebral foraminal canal of an individual;
b. at least one recording electrode that is adapted to be positioned or implanted at a dorsal root of the individual;
c. a processor that is adapted to run a neural stimulation algorithm, wherein the at least one recording electrode is adapted to communicate sensed afferent neural activities to the processor, and wherein the neural stimulation algorithm is adapted to drive closed-loop, intelligent control of the one or more stimulating electrodes based at least in part on sensed information communicated by the at least one recording electrode;
wherein the electrical stimulation is delivered at an initial neuromodulation frequency that is selected at least in part based on conduction velocity of the one or more targeted afferents.

2. The system of claim 1, wherein the initial neuromodulation frequency is proportional to the conduction velocity of the one or more targeted afferents.

3. The system of claim 1, wherein the one or more afferents within the intervertebral foraminal canal are selected from the group consisting of a dorsal root, dorsal root ganglion (DRG), T-junction, spinal nerve and combinations thereof.

4. The system of claim 1, wherein the electrical stimulation delivers a temporal and spatial summation of multichannel stimulation.

5. The system of claim 1, wherein the one or more stimulating electrodes comprises at least two stimulating electrodes, and wherein the at least two stimulating electrodes deliver stimulation energy individually or simultaneously.

6. The system of claim 1, wherein the selective sensory transmission block is effected in a sub-population of unmyelinated C-fibers and slow-conducting Aδ fibers.

7. The system of claim 6, wherein the selective sensory transmission block functions to block slow-conducting C-fibers and Aδ-fibers to stop nociceptive signals from transmitting to the spinal cord, thereby facilitating treatment of pain arising from visceral organs that lack fast-conducting A fibers.

8. The system of claim 1, wherein the one or more stimulating electrodes comprise at least two stimulating electrode leads, and wherein the at least two stimulating electrode leads selectively block a sub-population of C-fibers and slow-conducting Aδ fibers by delivering combined stimulation at a T-junction for the sub-population of C-fibers and slow-conducting Aδ fibers from the at least two stimulating electrode leads, each of which delivers stimuli at a frequency below the initial neuromodulation frequency that would be selected in the absence of a plurality of stimulating electrode leads.

9. The system of claim 1, wherein the algorithm is adapted to automatically initiate neuromodulation to block abnormal neural activity and to adjust the stimulus intensity and frequency after transmission block is achieved to improve efficiency of the neuromodulation regimen.

10. The system of claim 1, wherein the initial neuromodulation frequency is less than about 5 Hz.

11. A method for electrical stimulation to effectuate selective sensory transmission block, comprising:
a. providing one or more stimulating electrodes adapted to deliver electrical simulation to one or more targeted afferents within an intervertebral foraminal canal of an individual;
b. providing at least one recording electrode that is adapted to be positioned or implanted at a dorsal root of the individual;
c. a processor that is adapted to run a neural stimulation algorithm, wherein the at least one recording electrode is adapted to communicate sensed afferent neural activities to the processor, and wherein the neural stimulation algorithm is adapted to drive closed-loop, intelligent control of the one or more stimulating electrodes based at least in part on sensed information communicated by the at least one recording electrode;
wherein the electrical stimulation is delivered at an initial neuromodulation frequency that is selected at least in part based on conduction velocity of the one or more targeted afferents.
